(19) United States
(12) Patent Application Publication
(10) Pub. No.: US 2026/0199406 A1
(43) Pub. Date: Jul. 16, 2026

(54) LOWER AIRWAY PROBIOTICS AND METHODS OF USE FOR PROTECTION AND TREATMENT OF RESPIRATORY DISEASE

(71) Applicants: Adam P. Arkin, Oakland, CA (US); Kelsey E. Hern, Oakland, CA (US); Steven S. Branda, Albuquerque, NM (US); THE REGENTS OF THE UNIVERSITY OF CALIFORNIA, Oakland, CA (US); NATIONAL TECHNOLOGY & ENGINEERING SOLUTIONS OF SANDIA, LLC, Albuquerque, NM (US)

(72) Inventors: Adam P. Arkin, Oakland, CA (US); Kelsey E. Hern, Oakland, CA (US); Steven S. Branda, Albuquerque, NM (US)

(73) Assignees: THE REGENTS OF THE UNIVERSITY OF CALIFORNIA, Oakland, CA (US); NATIONAL TECHNOLOGY & ENGINEERING SOLUTIONS OF SANDIA, LLC, Albuquerque, NM (US)

(21) Appl. No.: 19/136,724

(22) PCT Filed: Dec. 8, 2023
(86) PCT No.: PCT/US2023/083196

§ 371 Date: Jun. 6, 2025

Related U.S. Application Data
(60) Provisional application No. 63/431,301, filed on Dec. 8, 2022.

(51) Int. Cl. A61K 35/741; A61K 9/00; A61K 35/00; A61K 35/742; A61P 31/04

(52) U.S. Cl. CPC A61K 35/741; A61K 9/0075; A61K 9/0078; A61K 35/742; A61P 31/04; A61K 2035/115

(57) ABSTRACT

In certain embodiments, the present invention provides a pharmaceutical composition comprising a pharmaceutically acceptable carrier and a lower airway probiotic composition comprising a physiological solution and a single candidate probiotic (CP) population, wherein the CP is CP4, CP7, CP8, CP9, CP13, CP17, CP18, CP19, CP20, or CP26. In certain embodiments, the present invention provides a pharmaceutical composition comprising a pharmaceutically acceptable carrier and a lower airway probiotic composition, wherein the lower airway probiotic composition comprises at least two candidate probiotic (CP) populations CP8 and CP19, CP20 and CP17, or CP19 and CP26 Also provided are kits and methods of using these pharmaceutical compositions.

### CROSS-REFERENCE TO RELATED APPLICATION

This application claims priority to U.S. Provisional Application No. 63/431,301, filed Dec. 8, 2022, the contents of which are incorporated by reference herein.

### STATEMENT REGARDING FEDERALLY SPONSORED RESEARCH

This invention was made with government support under DE-NA0003525 awarded by U.S. Department of Energy. The government has certain rights in the invention.

### BACKGROUND

Interest in the role of microbiota in human and animal health has grown significantly. Previously, the lower airway including the lung was considered to be a sterile environment. More recently, it has been demonstrated that colonization by complex population of microorganisms in the lower airways also occurs in healthy subjects.

The “microbiota” is made up of different species of microorganisms that live in a defined environment. In an animal body, microbiota is present in organs that come into contact with the outside environment, in the gut and also in the lung. The microbiota differs among various individuals and also varies according to pathological events or the individual's health state, and it can modulate immune response. The term “microbiome” is considered to include the complete set of microorganisms (bacteria, viruses, and fungi) and their genomes. There are numerous mutually beneficial interactions between the host body and microbiota with metabolic reactions, which are important for health and can contribute to the pathogenesis of some diseases.

It is believed that the upper airway microbiota is the primary contributor to the lower airway microbiota. The healthy lung is colonized only by a limited population of bacteria that are maintained by an equilibrium among immigration, elimination, and growth. Actinobacteria, Proteobacteria, Bacteroidetes, and Firmicutes ribosomal DNA has been shown to exist in healthy people's lungs. Some changes in the local environment during pathology can permit an increase in some bacterial population that could become pathological, especially in chronic conditions. Aspiration of oropharyngeal secretions, micro aspiration, or direct dispersal by contagious mucosa can create the microbiome environment in the lung.

The microbiota is directly connected to the immune system, and it undergoes metabolic and antigenic interactions. Dysbiosis in the gut microbiota is associated with lung disease because the microbiota participates in the development and maintenance of the immune system. Because dysbiosis can permit disease development, immunity can also influence the microbiota composition, which provides resistance to colonization by respiratory pathogens that have a reciprocal influence on maturation and health maintenance. The immune function derived by immune system homeostasis represents the mucosal barrier and its microbiome environment interactions in the intact barrier surface; when this unit is disrupted, immunity is compromised for a long time after an acute infection.

Accordingly, improved lower airway probiotics are needed.

### SUMMARY

In one aspect, provided herein is a pharmaceutical composition comprising a pharmaceutically acceptable carrier and a lower airway probiotic composition comprising a physiological solution and a single candidate probiotic (CP) population, wherein the CP is CP4, CP7, CP8, CP9, CP13, CP17, CP18, CP19, CP20, or CP26. In certain aspects, the CP is CP17. In certain aspects, the CP is CP19.

In one aspect, provided herein is a pharmaceutical composition comprising a pharmaceutically acceptable carrier and a lower airway probiotic composition, wherein the lower airway probiotic composition comprises at least two candidate probiotic (CP) populations CP8 and CP19, CP20 and CP17, or CP19 and CP26. In certain aspects, the CP population consists of CP8 and CP19. In certain aspects, the CP population consists of CP20 and CP17. In certain aspects, the CP population consists of CP19 and CP26.

In one aspect, provided herein is an inhaler comprising a pharmaceutical composition comprising a pharmaceutically acceptable carrier and a lower airway probiotic composition comprising a physiological solution and a single candidate probiotic (CP) population, wherein the CP is CP4, CP7, CP8, CP9, CP13, CP17, CP18, CP19, CP20, or CP26. In certain aspects, the CP is CP17. In certain aspects, the CP is CP19.

In one aspect, provided herein is an inhaler comprising a pharmaceutically acceptable carrier and a lower airway probiotic composition, wherein the lower airway probiotic composition comprises at least two candidate probiotic (CP) populations CP8 and CP19, CP20 and CP17, or CP19 and CP26. In certain aspects, the CP population consists of CP8 and CP19. In certain aspects, the CP population consists of CP20 and CP17. In certain aspects, the CP population consists of CP19 and CP26.

In one aspect, provided herein is a kit comprising at least one container and the pharmaceutical composition comprising a pharmaceutically acceptable carrier and a lower airway probiotic composition comprising a physiological solution and a single candidate probiotic (CP) population, wherein the CP is CP4, CP7, CP8, CP9, CP13, CP17, CP18, CP19, CP20, or CP26, wherein the CP population reduces lower airway infection in a recipient mammal, and instructions for delivery of an effective amount of the composition to the mammal in need thereof. In certain aspects, the CP is CP17. In certain aspects, the CP is CP19. In one aspect, provided herein is a kit comprising at least one container and the pharmaceutical composition comprising a pharmaceutically acceptable carrier and comprising a pharmaceutically acceptable carrier and a lower airway probiotic composition, wherein the lower airway probiotic composition comprises at least two candidate probiotic (CP) populations CP8 and CP19, CP20 and CP17, or CP19 and CP26, wherein the CP population reduces lower airway infection in a recipient mammal, and instructions for delivery of an effective amount of the composition to the mammal in need thereof. In certain aspects, the CP population consists of CP8 and CP19. In certain aspects, the CP population consists of CP20 and CP17. In certain aspects, the CP population consists of CP19 and CP26.

In one aspect, provided herein is a method of preventing or treating a lower respiratory infection in a mammal in need thereof comprising administering an effective amount of the pharmaceutical composition comprising a physiological solution and a single candidate probiotic (CP) population, wherein the CP is CP4, CP7, CP8, CP9, CP13, CP17, CP18, CP19, CP20, or CP26 to the lungs of the mammal. In certain aspects, the CP is CP17. In certain aspects, the CP is CP19.

In one aspect, provided herein is a method of preventing or treating a lower respiratory infection in a mammal in need thereof comprising administering an effective amount of the pharmaceutical composition comprising a physiological solution and at least two candidate probiotic (CP) populations CP8 and CP19, CP20 and CP17, or CP19 and CP26 to the lungs of the mammal. In certain aspects, the CP population consists of CP8 and CP19. In certain aspects, the CP population consists of CP20 and CP17. In certain aspects, the CP population consists of CP19 and CP26.

### BRIEF DESCRIPTION OF DRAWINGS

FIGS. 1A-1G: (FIG. 1A) Schematic of probiotic isolation method. (FIG. 1B) Phylogenetic tree of probiotic panel used in this work. Probiotics are highlighted and are interspersed amongst publicly available genomes. The model pathogen (Burkholderia thailandensis E264) is very similar to the human pathogen Burkholderia pseudomallei and slightly less similar to a common pathogen in cystic fibrosis patients, Burkholderia cepacia. Burkholderia thailandensis E264) is an art-recognized model for the human pathogen Burkholderia pseudomallei. (FIG. 1C) IC50 curves for four of the isolates in this panel. Absolute IC50 is reported as well as the 95% confidence interval. (FIG. 1D) Inhibition curves for all isolate in LRM. Top (from left to right): CP4, CP7, CP8, CP9, CP13; Bottom (from left to right): CP17, CP18, CP20, CP19, CP26. (FIG. 1E) Growth promoters: Top (left to right): CP9, CP13; Bottom (left to right): CP20, CP26. (FIG. 1F) Growth inhibitors: Top (left to right): CP4, CP8, CP17; vBottom (left to right): CP7, CP18, CP19. (FIG. 1G) Enlarged plot of CP7 (predicted to have the highest jaccard coefficient) and smaller panel showing higher ratios. At a 1000:1 ratio there is a 3-orders of magnitude reduction in B. thailandensis CFU counts.

FIGS. 2A-2D. (FIG. 2A) Matrix of jaccard fractions for all probiotics in the panel (FIG. 2B) Matrix of jaccard fractions excluding probiotics that are unable to colonize the lung (FIG. 2C) IC50 curves for two probiotics CP19 and CP8 individually against the pathogen and a 1:1 combination of the two (red). (FIG. 2D) IC50 curve of CP19 with or without the addition of 43 mM glucose—a rightward shift of the curve with the addition of glucose indicates that in the environment of the lung, carbon is being competed for.

FIGS. 3A-3B. (FIG. 3A) Lung colonization of two isolates CP17 and CP19 in the lung of mice 7 days after intranasal administration. (FIG. 3B) Survival curves for mice challenged with B. thailandensis infection with no prophylactic probiotic administration (top) prophylactic administration of CP19 (middle) or CP17 (bottom).

FIG. 4. CP combinations ordered from predicted synergistic (top) moderate synergy (bottom left) and no synergy (bottom right). It can be seen that jaccard fraction predicts three types of interactions, synergistic, non-additive (bottom left) and additive interactions (bottom right).

FIG. 5. qPCR data used to track growth of pathogen and two isolates when grown in mixed culture.

FIGS. 6A-6C. Characterization of CP activity against B. thailadensis in vitro. FIG. 6A. Phylogenomic tree of CPs and model pathogen B. thailandensis interspersed amongst publicly available genomes. FIG. 6B. Schematic of competition assay used to test in vitro inhibitory capabilities of CPs. Percent growth of Bt is calculated as the CFU in co-culture/CFU in Bt only culture multiplied by 100. Co-culture inhibition was tested and calculated for multiple starting densities of CP (see, FIG. 6C, x-axis). FIG. 6C. Relative IC50 curves for CPs against B. thailandensis. Relative IC50 is denoted with a dashed-dotted line and 100% and 50% growth marked with dotted lines.

FIGS. 7A-7D. Specialized metabolites play a role in anti-pathogen activities of CPS. FIG. 7A. Schematic representation of supernatant workflow. FIG. 7B. (left) Heatmap of fold-change in max DFU from supernatant of each inhibitory CP relative to B. thailadensis grown in supernatant from itself. FIG. 7C. Overlay assays of wildtype (WT) CP8 and sfp mutant (Δsfp). FIG. 7D. Dose-response curve of wildtype CP8 and sfp mutant against B. thailadensis.

FIGS. 8A-8I. Niche overlap is the primary mode of inhibition for CPs. FIG. 8A. Dose-response curve for CP7 grown in unsupplemented LRM (black) or with additional glucose (gray). FIG. 8B. Activity of CP7 against Bt at a 1:10 with or without glucose supplementation. FIG. 8C. Carbon utilization heatmap for all DPs in the panel and Bt. FIG. 8D. Venn diagram of carbon sources utilized by CP7 and Bt. FIG. 8E. Correlation between average normalized growth of t at a 1:1 ration and jaccard coefficient. FIG. 8F. Total carbon consumed by 3 isolates grown in combination with Bt at 36 hours. FIG. 8G. Total electron flux at 24 hours, gray dashed line indicates the estimated abundance of Bt in each co-culture. FIG. 8H. Electron flux of lactate (black), proline (white) and aspartic acid (gray) at 24 hurs in different isolate co-culture conditions. FIG. 8I. Receiver operator characteristic curve showing the ability of growth on lactate to distinguish inhibitory CPs form non-inhibitory CPs. An AUROC of 1 indicates perfect distinction, while the dashed line represents an a URPC of 0.5 indicating no distinction between inhibitors and non-inhibitors.

FIGS. 9A-9I. Niche overlap aids in identification of efficacious combinations. FIG. 9A. Venn diagram depicting niche index fraction calculation. The fraction numerator is the light-colored triangular shape at the intersection of CPx and CPy but does not intersect with Bt; and dark, center triangle is the denominator. FIG. 9B, Heatmap of niche index fractions for each colonizing CP. FIG. 9C, Dose-response curve for a combination of CP8/CP19 (diamonds-combination, triangles-CP19, Circles-CP8). FIG. 9D. Dose-response curve for a combination of CP19/CP13 (diamonds-combination, triangles-CP19, Circles-CP13). FIG. 9E. Dose-response curve for a combination of CP13/CP8 (diamonds-combination, triangles-CP8, Circles-CP13). FIG. 9F. Bliss independence calculation for the CP8/CP19 combination, dashed line indicates the expected effect for CP19 and CP8 used in combination. Calculated values above the line represent synergy and below the line, antagonism. FIG. 9G. Lactate consumed determined via GC-TOF for 3 conditions normalized to CFU of each CP inoculated. FIG. 9H. Dose-response curve for a combination of CP8/CP19 and CP8sfp/CP19. FIG. 9I. Proposed mechanism for CP8/CP19 combination inhibition. CP8 produces a specialized metabolite with inhibits growth of the pathogen, increase lactate consumption by CP19 in the presence of CP8 also creates nutrient competition with Bt. Overall, little niche overlap between eh CPs and high overlap with Bt allows further inhibition of the pathogen with minimal inter-CP competition.

FIGS. 10A-10E. CPs enhance survival in vivo. FIG. 10A. Schematic of engraftment testing for CPs. 10⁶ of CFU of each CP is administered via OPA and lungs are removed for CFU quantification after 7 days. FIG. 10B. Engraftment results from homogenized lung tissue from 7 days after CP administration. The dotted line represents the minimum CFU at which a CP is considered able to engraft. FIG. 10C. Schematic of method for survival testing Mice were administered 10⁶ CFU of each CP and then challenged with pathogen at days 3, 5 or 7. Survival was measured for 10 days following challenge. FIG. 10D. Survival data from mice administered sham saline control. FIG. 10E. Survival data from mice administered CPs 3 days, 5 days or 7 days prior to pathogen challenge. Niche index values are listed in the lower right corner for each CP.

FIG. 11. Proposed mechanism of CP protection. During lung infection (left) bacterial growth elicits inflammation from the host with concurrent endothelial and epithelial injury. Intra-alveolar oedema introduces additional nutrients from the blood into the lung alveoli and lumen allowing for increased bacterial growth of the pathogen. This positive feedback loop allows uncontrolled growth of the pathogen ultimately facilitating instantiation of infection. We propose that probiotics delivered directly to the lung may limit infection by reducing nutrient abundance after oedema (right). Limited nutrient abundance inhibits bacterial pathogen growth and rapidly ends the infection-promoting feedback loop.

FIG. 12. Growth curves of all CPs and Bt in LRM. Candidate probiotics and Bt were inoculated at 10⁶ CFU/ml into LSM. Cultures were grown for 48 hours.

FIG. 13. Addition of glucose decreases competition between CP19 and Bt. Dose-responsive curve of Bt in co-culture with CP19 in the presence of 5.5 mM glucose (dark line) or 43 mM glucose (light line). Vertical dotted lines represent the IC50 in the low or high glucose condition.

FIG. 14. Robust regression analysis for activity and niche index of all CPs excluding CP8. Excluded candidate probiotic CP26 is labeled.

FIGS. 15A-15B. Relative IC50 Curves for pairwise combinations with low niche index fractions. FIG. 15A. ID50 curve for combination of CP20 and CP17 (stars). FIG. 15B. ID50 curve for combination of CP26 and CP19 (stars).

FIG. 16. pPCR generated growth curves for CP8, CP19 and Bt grown in co-culture.

### DETAILED DESCRIPTION

Lower airway probiotics have developed for protection against respiratory disease. Specifically, host-mined probiotics can be administered directly to the lung for protection against a respiratory pathogen, Burkholderia thailandensis. Additionally, an in vitro pipeline has been developed for prediction of efficacy of each of probiotics, and combinations of such, in defense against the pathogen. Lower airway probiotics have been demonstrated as useful for protection against bacterial infections.

### Pharmaceutical Compositions

In certain aspects, the present invention provides a pharmaceutical composition comprising a pharmaceutically acceptable carrier and a lower airway probiotic composition comprising a physiological solution and a single candidate probiotic (CP) population, wherein the CP is CP4, CP7, CP8, CP9, CP13, CP17, CP18, CP19, CP20, or CP26. In certain aspects, the CP is CP17. In certain aspects, the CP is CP19.

In certain aspects, the present invention provides pharmaceutical composition comprising a pharmaceutically acceptable carrier and a lower airway probiotic composition, wherein the lower airway probiotic composition comprises at least two candidate probiotic (CP) populations CP8 and CP19, CP20 and CP17, or CP19 and CP26. In certain aspects, the CP population consists of CP8 and CP19. In certain aspects, the CP population consists of CP20 and CP17. In certain aspects, the CP population consists of CP19 and CP26.

In certain aspects, the pharmaceutical composition is formulated for administration to the lower airway of a subject.

In certain aspects, the pharmaceutical composition is formulated for administration to the respiratory tract of a subject by dry powder inhaler or a nebulizer.

In certain aspects, the pharmaceutically acceptable carrier is saline, hypertonic saline, or water. In certain aspects, the pharmaceutically acceptable carrier is saline.

In certain aspects, the pharmaceutical composition is useful for treating or preventing lower airway infection. In certain aspects, the infection to be treated or prevented is characterized by unwanted or overgrowth of microorganisms comprising Pseudomonas aeruginosa, Mycobacteria spp, Aspergillus spp, Staphylococcus aureus, Achromobacter spp, Stenophomonas maltophilia, Burkholderia spp, Haemophilus spp, Streptococcus pneumoniae or any combination thereof.

### Devices and Kits

In certain aspects, the present invention provides an inhaler comprising a pharmaceutical composition comprising a pharmaceutically acceptable carrier and a lower airway probiotic composition comprising a physiological solution and a single candidate probiotic (CP) population, wherein the CP is CP4, CP7, CP8, CP9, CP13, CP17, CP18, CP19, CP20, or CP26. In certain aspects, the CP is CP17. In certain aspects, the CP is CP19.

In certain aspects, the present invention provides an inhaler comprising a pharmaceutically acceptable carrier and a lower airway probiotic composition, wherein the lower airway probiotic composition comprises at least two candidate probiotic (CP) populations CP8 and CP19, CP20 and CP17, or CP19 and CP26. In certain aspects, the CP population consists of CP8 and CP19. In certain aspects, the CP population consists of CP20 and CP17. In certain aspects, the CP population consists of CP19 and CP26.

In certain aspects, the present invention provides a kit comprising at least one container and the pharmaceutical composition comprising a pharmaceutically acceptable carrier and a lower airway probiotic composition comprising a physiological solution and a single candidate probiotic (CP) population, wherein the CP is CP4, CP7, CP8, CP9, CP13, CP17, CP18, CP19, CP20, or CP26, wherein the CP population reduces lower airway infection in a recipient mammal, and instructions for delivery of an effective amount of the composition to the mammal in need thereof. In certain aspects, the CP is CP17. In certain aspects, the CP is CP19.

In certain aspects, the present invention provides a kit comprising at least one container and the pharmaceutical composition comprising a pharmaceutically acceptable carrier and comprising a pharmaceutically acceptable carrier and a lower airway probiotic composition, wherein the lower airway probiotic composition comprises at least two candidate probiotic (CP) populations CP8 and CP19, CP20 and CP17, or CP19 and CP26, wherein the CP population reduces lower airway infection in a recipient mammal, and instructions for delivery of an effective amount of the composition to the mammal in need thereof. In certain aspects, the CP population consists of CP8 and CP19. In certain aspects, the CP population consists of CP20 and CP17. In certain aspects, the CP population consists of CP19 and CP26.

In certain aspects, the kit further contains an inhaler or nebulizer.

### Modes of Administration

In one aspect, provided herein is a method of preventing or treating a lower respiratory infection in a mammal in need thereof comprising administering an effective amount of the pharmaceutical composition comprising a physiological solution and a single candidate probiotic (CP) population, wherein the CP is CP4, CP7, CP8, CP9, CP13, CP17, CP18, CP19, CP20, or CP26 to the lungs of the mammal. In certain aspects, the CP is CP17. In certain aspects, the CP is CP19.

In one aspect, provided herein is a method of preventing or treating a lower respiratory infection in a mammal in need thereof comprising administering an effective amount of the pharmaceutical composition comprising a physiological solution and at least two candidate probiotic (CP) populations CP8 and CP19, CP20 and CP17, or CP19 and CP26 to the lungs of the mammal. In certain aspects, the CP population consists of CP8 and CP19. In certain aspects, the CP population consists of CP20 and CP17. In certain aspects, the CP population consists of CP19 and CP26.

In certain aspects, the pharmaceutical composition is administered by means of a nebulizer. In certain aspects, the pharmaceutical composition is administered by means of a dry powder inhaler.

In certain aspects, the effective amount of the CP comprises between about 0.9×10⁶ CFU to about 36.9×10⁶ CFU per day.

In certain aspects, the CP is administered at a concentration of 10⁶ CFU (colony forming units).

In certain aspects, the infection is Burkholderia pseudomallei.

In certain aspects, the mammal is a human.

The terms “administering” and “administration” as used herein refer to a process by which a therapeutically effective amount of a compound of the disclosure or compositions contemplated herein are delivered to a subject for prevention and/or treatment purposes. Compositions are administered in accordance with good medical practices taking into account the subject's clinical condition, the site and method of administration, dosage, patient age, sex, body weight, and other factors known to physicians.

The term “bacterial population” as used herein refers to a homogeneous population of bacteria consisting of a single strain of a bacterial species or to a heterogeneous population of bacteria consisting of at least two distinguishable strains of a single bacterial species or of a plurality of species.

The terms “co-administration” or “co-administered” as used herein refer to the administration of at least two compounds or agent(s) or therapies to a subject. In some embodiments, the co-administration of two or more agents/therapies is concurrent. In other embodiments, a first agent/therapy is administered prior to a second agent/therapy in this aspect, each component may be administered separately, but sufficiently close in time to provide the desired effect, in particular a beneficial, additive, or synergistic effect. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

The term “composition” as used herein refers to a product comprising the specified ingredients in the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure and a pharmaceutically acceptable carrier.

A composition of the disclosure can be a liquid solution, suspension, emulsion or a powder. Various delivery systems are known and can be used to administer a composition of the disclosure, e.g., encapsulation in liposomes, microparticles, microcapsules, and the like, and then delivered to a patient by means of such as a nebulizer.

Compositions for administration may include sterile aqueous or non-aqueous solvents, such as water, isotonic saline, isotonic glucose solution, buffer solution, or other solvents conveniently used for parenteral administration of therapeutically active agents, stabilizers, buffers, or preservatives, e.g., antioxidants such as methylhydroxybenzoate or similar additives.

A composition of the disclosure may be sterilized by, for example, addition of sterilizing agents to the composition, irradiation of the composition, or heating the composition. Alternatively, the compounds or compositions of the present disclosure may be provided as sterile solid preparations e.g., lyophilized powder, which are readily dissolved in sterile solvent immediately prior to use.

After pharmaceutical compositions have been prepared, they can be placed in an appropriate container and labeled for treatment of an indicated condition. For administration of a composition of the disclosure, such labeling would include amount, frequency, and method of administration.

The term “freeze-dried (lyophilized) as used herein refers to a preparation of bacterial cells that have been initially frozen and the water content removed by vacuum.

The terms “inactivated probiotic” or “inactivated micro-organism” as used herein mean that the metabolic activity or reproductive ability of the organism has been reduced or destroyed. The inactivated organisms do, however, still retain, at the cellular level, at least a portion their biological glycol-protein and DNA/RNA structure. As used herein, the term “inactivated” is synonymous with “nonviable”.

The term “pharmaceutically acceptable carrier” as used herein refers to a diluent, adjuvant, excipient, or vehicle with which a probe of the disclosure is administered and which is approved by a regulatory agency of the Federal or a state government or listed in the U.S. Pharmacopeia or other generally recognized pharmacopeia for use in animals, and more particularly in humans. Such pharmaceutical carriers can be liquids, such as water and oils, including those of petroleum, animal, plant or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. When administered to a patient, the probe and pharmaceutically acceptable carriers can be sterile. Saline solutions and aqueous dextrose and glycerol solutions can also be employed as liquid carriers, particularly for injectable solutions. Suitable pharmaceutical carriers also include excipients such as glucose, lactose, sucrose, glycerol monostearate, sodium chloride, glycerol, propylene, glycol, water, ethanol and the like. The present compositions, if desired, can also contain minor amounts of wetting or emulsifying agents, or pH buffering agents. The present compositions advantageously may take the form of solutions, emulsion, sustained-release formulations, or any other form suitable for use.

The term “probiotic” is recognized in the state of the art as a microorganism which, when administered in adequate amounts, confers a health benefit to the host. A probiotic microorganism must fulfil several requirements related to lack of toxicity, viability, adhesion and beneficial effects. These probiotic features are strain-dependent, even among bacteria of the same species. Therefore, it is important to find those strains that have a better performance in all probiotic requirements.

The terms “treating” or “treatment” as used herein refers to an alleviation of symptoms associated with a disorder or disease, or inhibition of further progression or worsening of those symptoms, or prevention or prophylaxis of the disease or disorder, or curing the disease or disorder. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms, or prevents or provides prophylaxis for the disorder or condition. In particular, a “therapeutically effective amount” refers to an amount of a therapy (e.g., a prophylactic or therapeutic agent) that is sufficient to reduce or ameliorate the severity and/or duration of a respiratory condition or one or more symptoms thereof, prevent the advancement of a respiratory condition, cause regression of a respiratory condition, prevent the recurrence, development, or onset of one or more symptoms associated with a respiratory condition, or enhance or improve the prophylactic or therapeutic effect(s) of another therapy (e.g., prophylactic or therapeutic agent).

“Naturally occurring” is used to describe an object that can be found in nature as distinct from being artificially produced. For example, a protein or nucleotide sequence present in an organism (including a virus), which can be isolated from a source in nature and which has not been intentionally modified by man in the laboratory, is naturally occurring.

“Wild-type” refers to the normal gene, or organism found in nature without any known mutation.

“Genome” refers to the complete genetic material of an organism.

“Naturally occurring,” “native,” or “wild-type” is used to describe an object that can be found in nature as distinct from being artificially produced. For example, a protein or nucleotide sequence present in an organism (including a virus), which can be isolated from a source in nature and that has not been intentionally modified by a person in the laboratory, is naturally occurring.

The invention will now be illustrated by the following non-limiting Examples.

### Example 1

### Host-Mined Lower Airway Probiotics for Protection and Treatment of Respiratory Disease

As antibiotic resistance rises, there is increasing need for the development of technologies for treatment and prevention of infection. For years, the respiratory microbiota have been theorized to be the gatekeepers of respiratory health as pathogens entering the lung make contact with the microbiota prior to or coincident to their interaction with the host cell. Probiotics administered directly to the lung are an attractive target for novel antimicrobials against respiratory pathogens as they are unlikely to be hindered by antibiotic resistance and are delivered exclusively at the site of infection potentially reducing off-target effects.

While lower airway probiotics (LAPs) are an attractive target for development of novel antimicrobials, none have yet demonstrated the ability of host-mined probiotics to protect against bacterial infection. In the present work, it is demonstrated that not only can several of these probiotics prolong survival in mice infected with Bacillus thailandensis, it is also shown that an in vitro system can be used to systematically predict which individual organisms and combinations will be most efficacious.

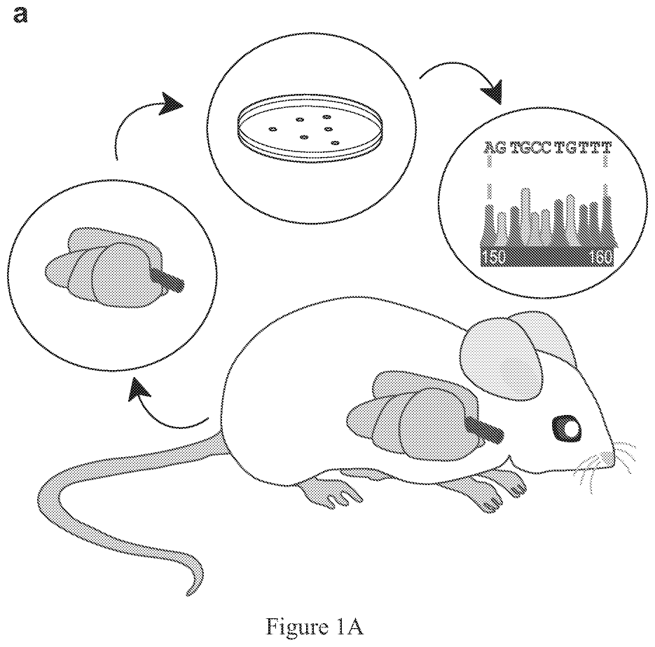

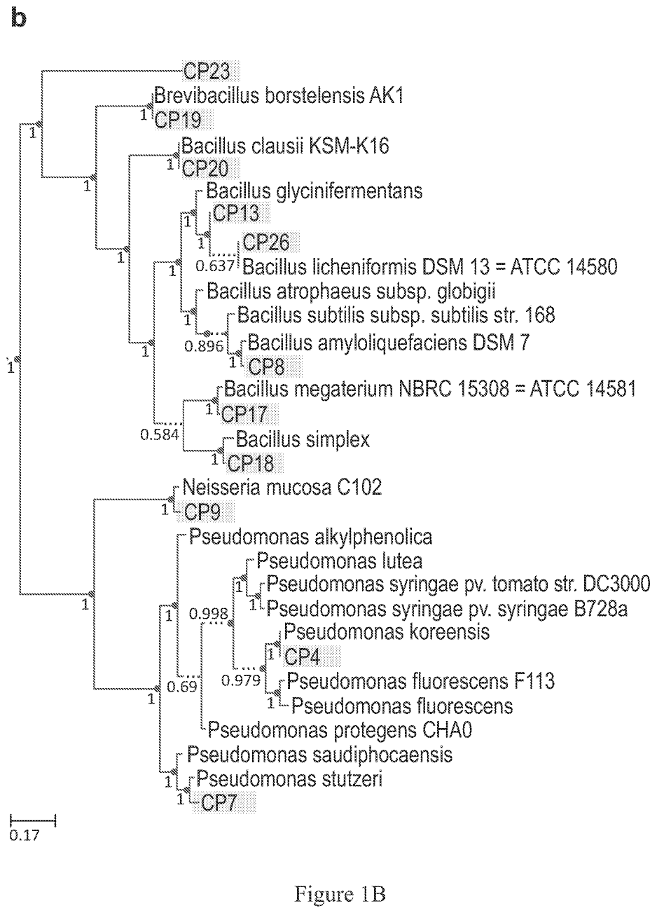

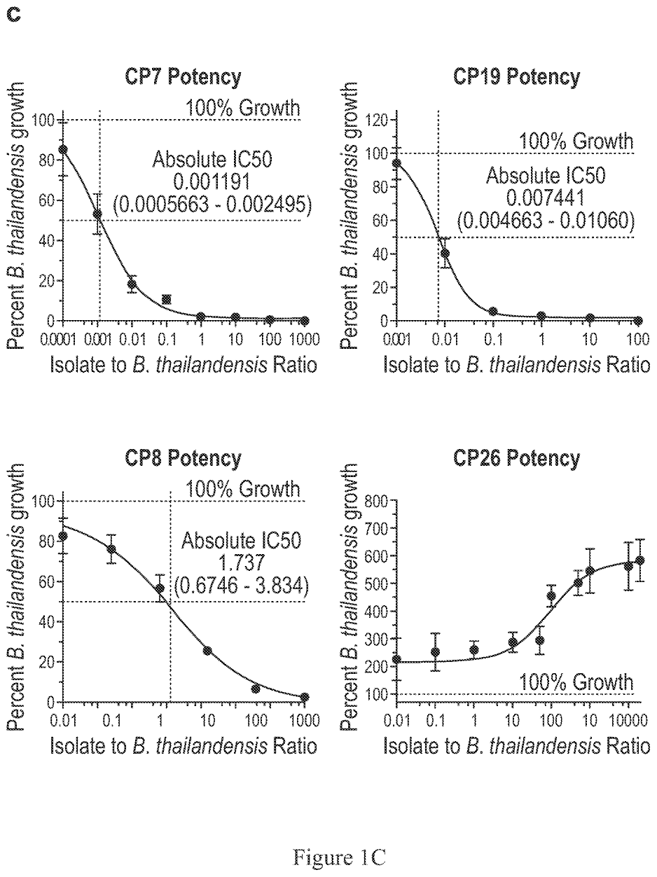

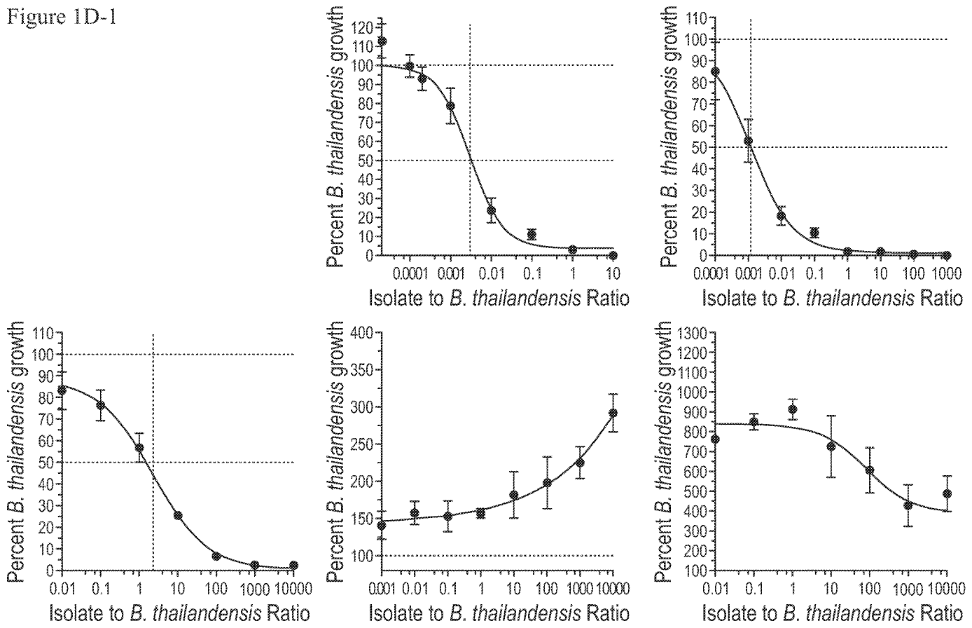

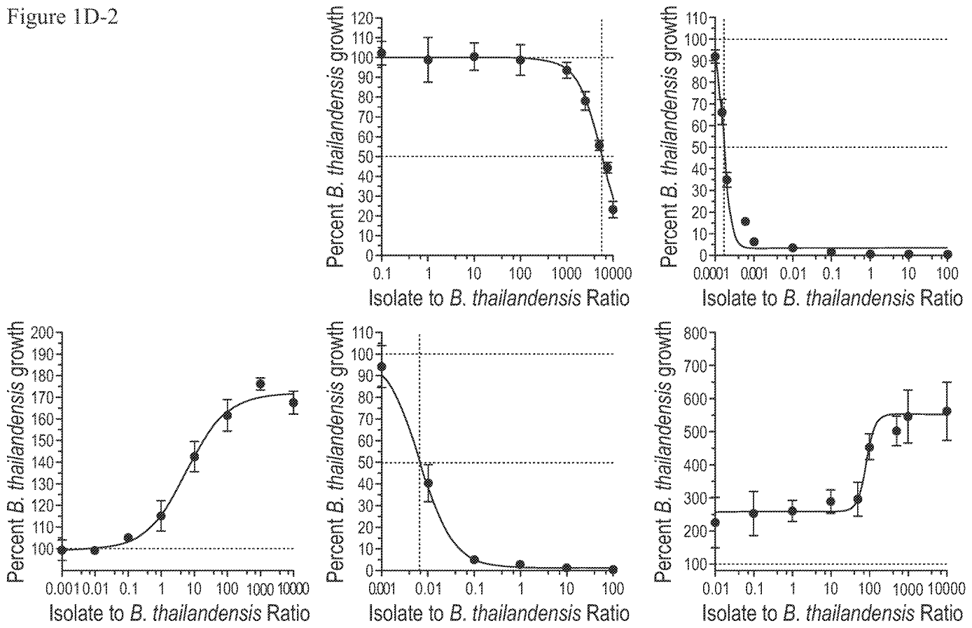

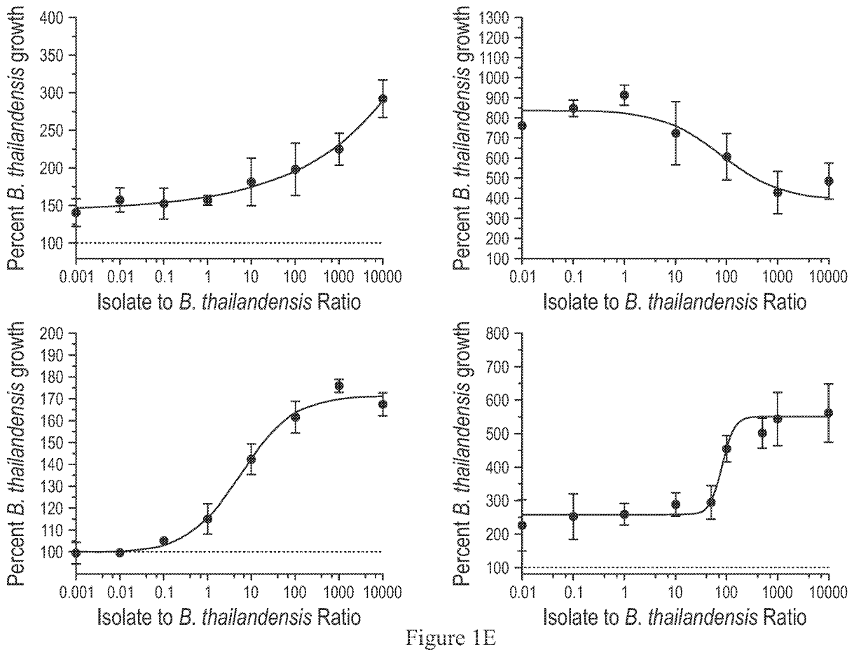

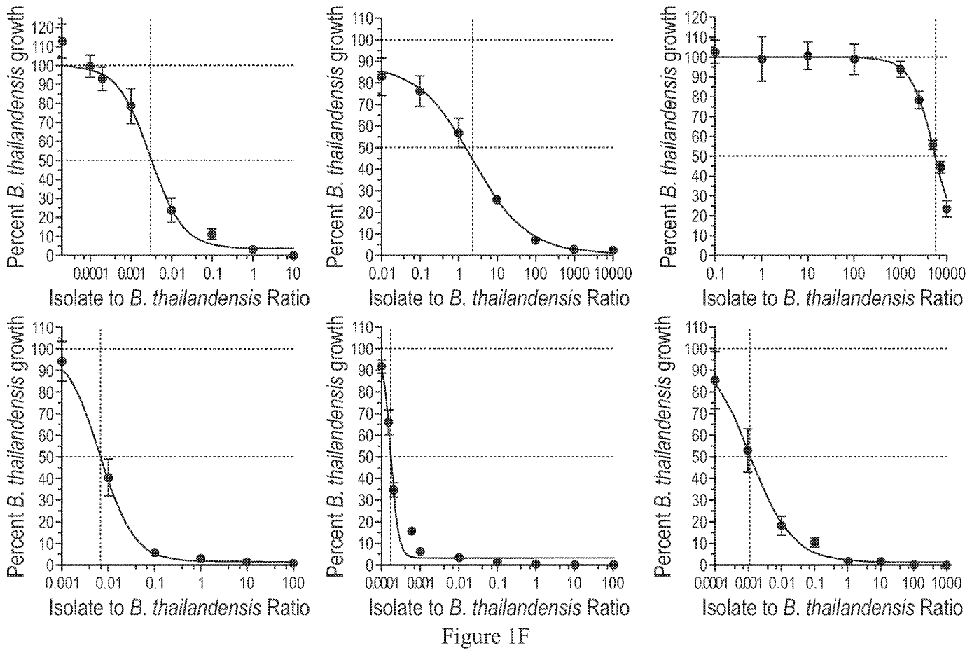

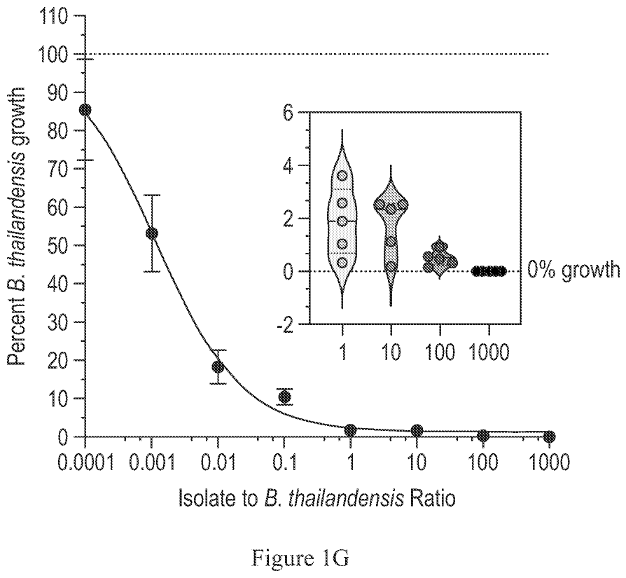

To achieve this, native lung organisms were isolated from healthy mice (FIGS. 1A and 1B). In order to determine the potency of each isolate against the pathogen, a platform was developed for testing their direct competition with the pathogen. To do this, increasing concentrations of isolate are co-cultured with the pathogen in a lung replicating medium, which is called synthetic sputum medium or sSM. This medium is also called lung-replicating medium (LRM) (Palmer K L, Aye L M, Whiteley M. J Bacteriol. 2007 November; 189(22):8079-87. doi: 10.1128/JB.01138-07. Epub 2007 Sep. 14). B. thailandensis growth is quantified for each concentration of isolate and used to generate an inhibitory concentration 50 (IC50) curve and value (FIGS. 1C and 1D). This value is the measure of the potency of the isolate where a lower IC50 indicates a more potent inhibitor of the pathogen (Table 1).

TABLE 1
Re relative IC50 values and 95% confidence intervals.

| | CP4 | CP7 | CP8 | CP17 | CP18 | CP19 |
|---|---|---|---|---|---|---|
| Relative IC50 | 0.003086 | 0.001172 | 2.316 | 5738 | 0.0001693 | 0.006604 |
| (95% CI) | (0.001791- | (.0006442- | (0.8280- | (3598- | (0.0001633- | (0.004842- |
| | 0.005331) | 0.003346) | 5.073) | ND) | 0.0001850) | 0.01141) |

Certain growth promoters were evaluated (FIG. 1E), as were growth inhibitors (FIG. 1F). CP7 had the highest jaccard coefficient (FIG. 1G).

A measure of niche overlap, termed a jaccard coefficient, is also used to predict potency against the pathogen. The term “Jsputum” describes the niche overlap between two organisms specifically in the environment of the lung. To do this, a weighted niche score is inputted for each organism (Aₛₚᵤₜᵤₘ or Bₛₚᵤₜᵤₘ), which is described by the number of carbon sources consumed by that organism and the relative energy contribution of each of those sources as measured by the abundance of and potential electron contribution of each source:

$${A}_{sputum =} \sum {C}_{utilized} \times {W}_{e lectrons}$$

These inputs can be used to calculate the overlap between each probiotic in the panel and the pathogen:

$${J}_{sputum} = \frac{\vert {A}_{sputum} ⋂ {B}_{sputum} \vert}{\vert {A}_{sputum} ⋃ {B}_{sputum} \vert}$$

As the Jsputum approaches 1, there is increasing overlap between the pathogen and the probiotic suggesting that competition and thus protection conferred by the probiotic may be increased (Table 2).

TABLE 2
Lung-specific Jaccard coefficients generated for each
isolate (Aₛₚᵤₜᵤₘ) with the pathogen (Bₛₚᵤₜᵤₘ)

| Isolate | CP4 | CP7 | CP19 | CP17 | CP18 | CP20 | CP26 | CP9 | CP13 | CP8 |
|---|---|---|---|---|---|---|---|---|---|---|
| J(Aₛₚᵤₜᵤₘ, Bₛₚᵤₜᵤₘ) | 0.966 | 0.944 | 0.883 | 0.800 | 0.722 | 0.679 | 0.630 | 0.617 | 0.615 | 0.599 |

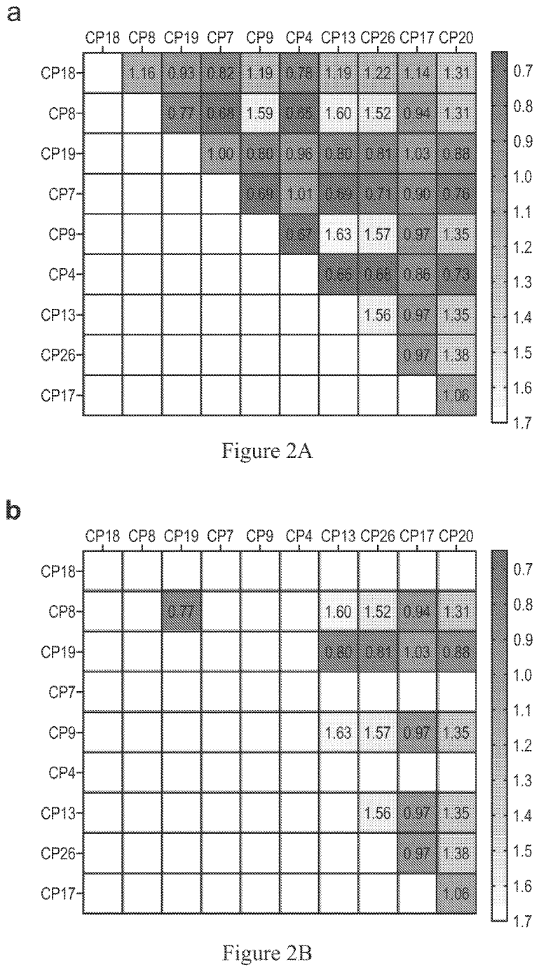

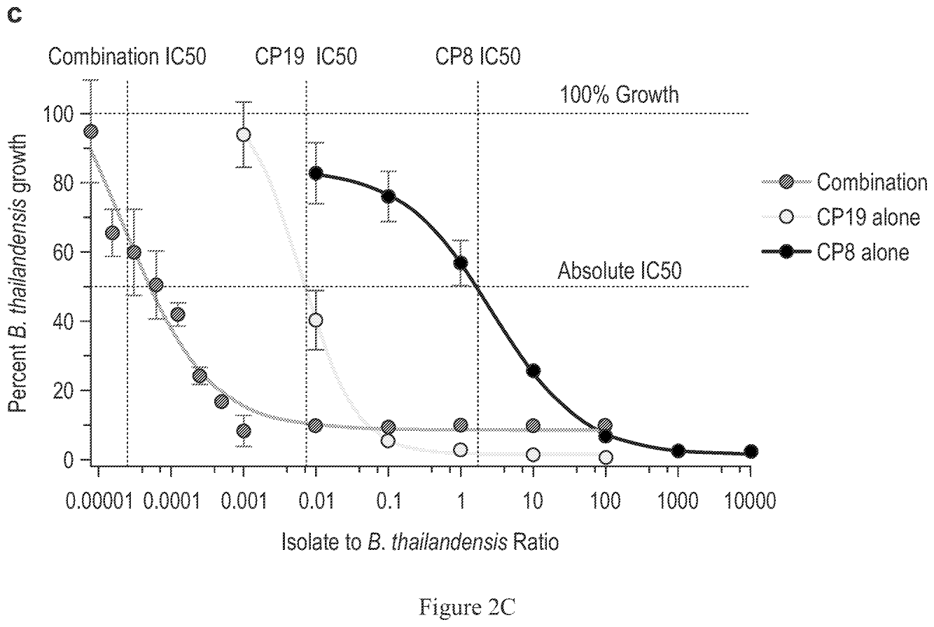

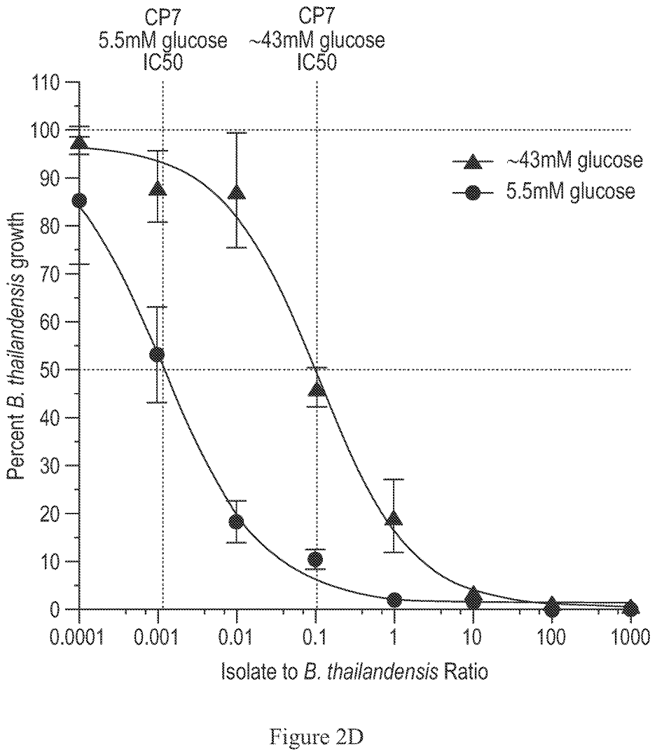

Even in the area of gut-delivered probiotic research, it has been difficult to identify probiotic cocktails in vitro that demonstrate synergistic effects in vivo. In order to create a metric for identifying synergies between respiratory isolates, the “Jaccard fraction” was created. The numerator of the Jaccard fraction represents the niche overlap between all the probiotics in a given combination and the denominator, the niche overlap between the combination and the pathogen. Synergies are most likely to occur in those combinations with a low Jaccard fraction. To identify strong synergies in vitro, a Jaccard fraction was generate for every possible combination of probiotics in the present panel (FIG. 2A).

TABLE 3
IC50 Values for Combinations

| | CP20/ | CP19/ | CP8/ | CP19/ | CP8/ |
|---|---|---|---|---|---|
| | CP17 | CP26 | CP19 | CP13 | CP13 |
| Relative | 1501 | 3.52 × 10⁻⁵ | 4.707 × 10⁻⁵ | 0.002126 | ND |
| IC50 | (1071- | (2.57 × 10⁻⁵- | (3.256 × 10⁻⁵- | (0.0007314- | |
| (95% CI) | 1935) | 5.105 × 10⁻⁵) | 9.914 × 10⁻⁵) | 0.008040) | |

After eliminating combinations containing isolates that do not colonize the mouse well, one combination CP19 (Brevibacillus borstelensis) and CP8 (Bacillus velezensis) appeared to have the lowest Jaccard fraction. When an IC50 curve is generated for this combination, the IC50 is lower indicating that this combination acts synergistically to kill the pathogen (FIGS. 2B and 2C). CP19 was tested with or without the addition of 43 mM glucose—a rightward shift of the curve with the addition of glucose indicates that in the environment of the lung, carbon is being competed for (FIG. 2D).

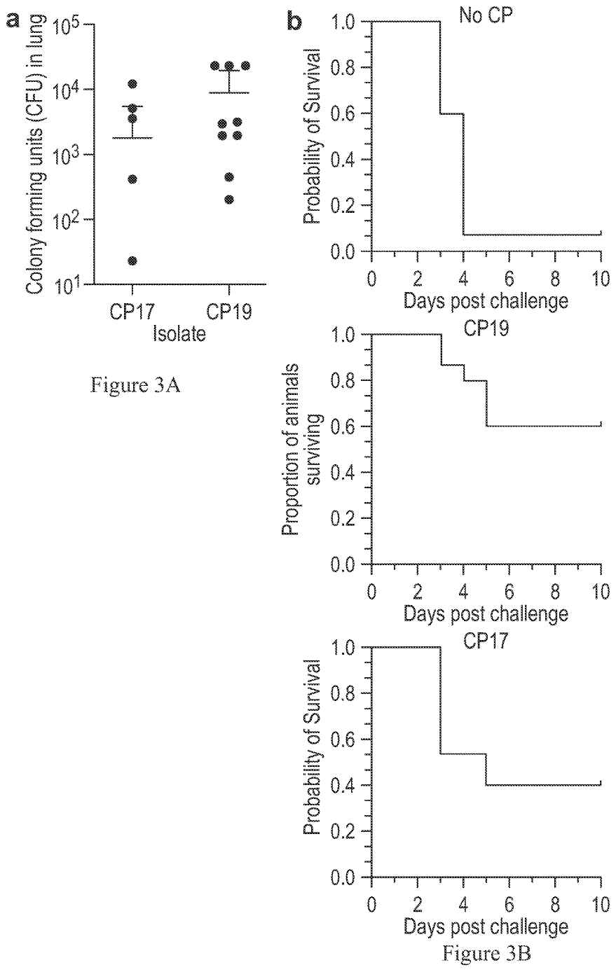

Finally, it was sought to verify that the probiotics perform as expected in vivo against the pathogen. To achieve this, probiotics were dosed 7 days prior to a challenge with B. thailandensis and survival was measured for 10 days. After 10 days, it was observed that two probiotics confer protection against the pathogen (FIGS. 3A-3B). These two isolates represent the two highest Jaccard coefficients of those probiotics that are able to colonize the lung.

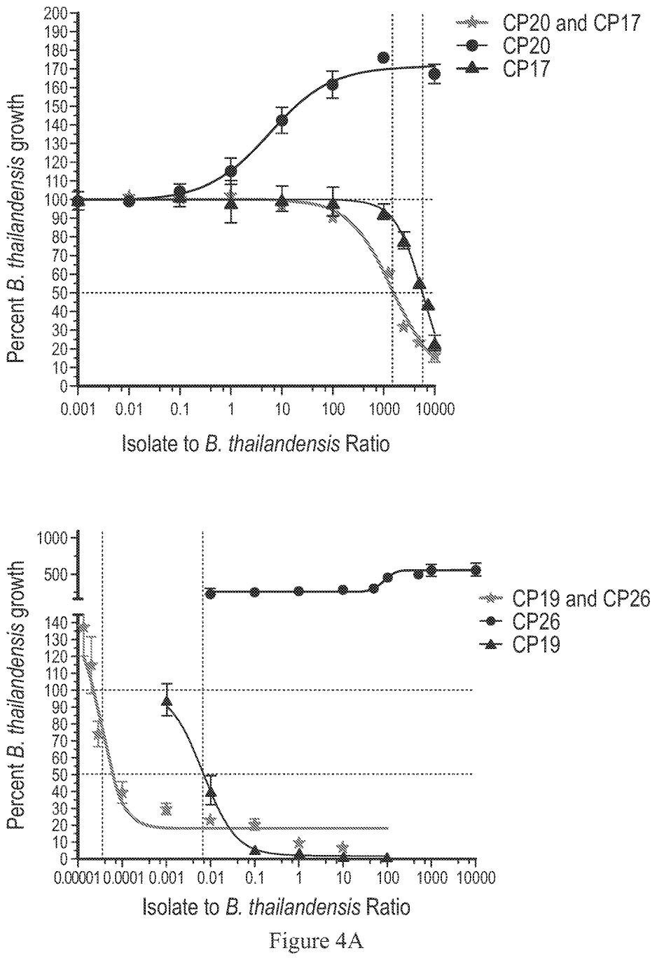

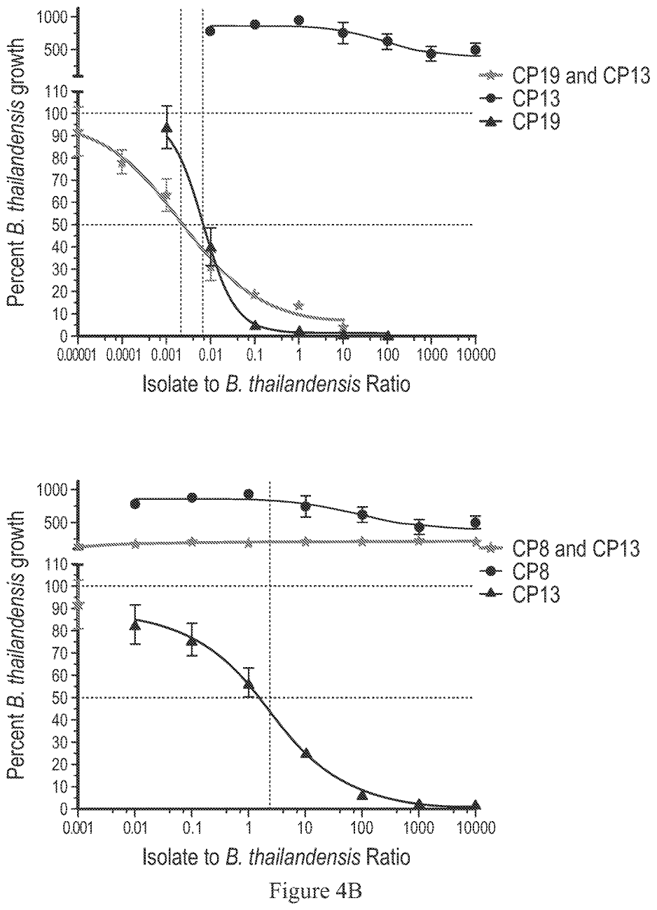

Further, CP combinations ordered from predicted synergistic, moderate synergy, and no synergy were observed (FIG. 4).

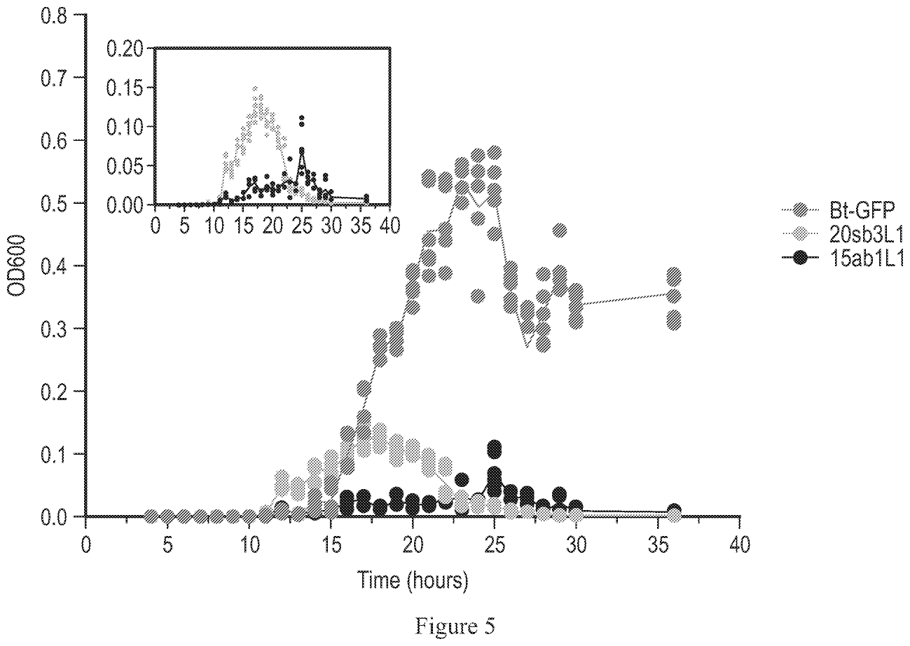

qPCR data used to track growth of pathogen and two isolates (CP8 and CP19) when grown in mixed culture, showed strong inhibition (FIG. 5).

As a whole, lower airway probiotics represent one of the few alternatives to antibiotics for prevention and treatment of respiratory infection. In comparison to antibiotics, LAPs are not likely to be affected by antibiotic resistance in their target organism and are capable of being used prophylactically in populations which are likely to experience certain diseases. In addition, LAPs are less likely to have off-target effects (e.g., gut dysbiosis) because they are administered exclusively to the site of disease. Finally, LAPs may prove to have better potency than antibiotics since they treat disease at the source of infection (i.e., the lung) and exist stably at their target site for 7 or more days unlike antibiotics whose concentration in the lung rises and declines with dosing schedule. Finally, in terms of development as therapies, the organisms used primarily in this work are classified as belonging to the Bacillus genera which has generally-regarded-as-safe status by the FDA making the pipeline to clinical use quicker.

Similar to antibiotics, LAPs may potentially outperform phage therapies as phages require repeat dosing in order to be effective and also cannot be used prophylactically. Additionally, identifying phage that are effective against a patient's particular pathogen can be time and labor intensive. Using the present platform, it was possible to easily identify two out of a panel of 10 probiotics that were effective in vivo.

No other comparable platforms exist for predicting efficacious probiotics in vivo as those described herein. This method has significant advantages over current methods of creating new probiotics which rely on costly and time-consuming in vivo screening methods to determine efficacy.

As currently described, this technology can be used to create novel probiotics and probiotic cocktails for prevention and treatment of infectious respiratory disease. In particular, the prediction pipeline is most useful in the development of therapies for bacterial infectious disease.

### Example 2

### Niche Exclusion of a Lung Pathogen in Mice with Designed Probiotic Communities

### Abstract:

For years, the lung microbiota have been theorized to be the gatekeepers of respiratory health as pathogens entering the lung make contact with the resident microbes prior to or coincident with their interaction with the host cell. Thus, tuning of the native lung community may present an opportunity to alter the state of the environment in favor of health. While probiotic supplementation to prevent pathogen infiltration has been explored extensively in the gut, little has been done to study this phenomenon in the lower respiratory tract. In this work, it was hypothesized that synthetic bacterial communities introduced into the lung can serve as prophylactic countermeasures against infection by a model pathogen, Burkholderia thailandensis, in mice. Here, it was demonstrated that understanding of antagonistic interactions between a pathogen and lung microbiota in vitro can guide the selection of probiotics with protective capabilities in vivo. While the production of secondary metabolites appears to play a role in pathogen antagonism, exploitative competition is the predominant mechanism deployed by these probiotics for pathogen defense. Specifically, it was shown that niche overlap and resource competition between the invading pathogen and probiotic are suggestive of performance in vivo.

### Introduction:

Every mammalian body compartment that opens to the outside world has microbial life associated with it. Until recently, the healthy lung was thought to be a sterile environment; however, recent work has demonstrated that the lower respiratory tract is colonized by a complex and roughly stable microbiome that impacts the health of the host. For years, the airway microbiota have been theorized to be gatekeepers of respiratory health, as pathogens entering the airway make contact with its microbiota prior to or coincident with their interactions with host cells. While the latter interactions have been studied in detail, little is known about pathogen-microbiome interactions in the airway.

Mechanistically, it has been hypothesized that pathogen establishment in the airway could be halted by its microbiota through either indirect or direct colonization resistance. During indirect competition, airway microbiota modulate the host immune response to increase surveillance, promoting rapid recognition and clearance of foreign microbe including the pathogen. During direct competition, the airway microbiota competitively exclude the pathogen through production of inhibitory specialized metabolites, or by occupying a nutritional niche that would otherwise be used by the pathogen. Occupation of a nutritional niche by airway microbiota may be particularly advantageous in those with inflammatory conditions, as inflammation generates metabolic waste products that can feed pathogen growth, potentially increasing risk of serious infection. Manipulation of the airway microbiome through use of probiotics (health-promoting microbes) could enhance protective activities and thereby prevent respiratory infection.

For example, patients with cystic fibrosis have an increased risk of acquiring infections by organisms of the Burkholderia cepacia complex (Bcc) which have both become increasingly resistant to current antibiotics. In particular, Bcc organisms have been highly associated with mortality in these populations for more than two decades. While the potential of prophylactic lung probiotics in cystic fibrosis patients for preventing Bcc infection is a promising alternative to antibiotics, current methods for probiotic development involve costly and time intensive in vitro and in vivo screening making them clinically unfeasible. To enable development of these therapies in the future, pipelines for probiotic nomination need to be made which can rapidly and cheaply identify efficacious probiotics. Further, systematic analysis of the mechanism of action of these probiotics would enable fine-tuned control of their activity allowing for more reliable therapeutics.

Here, it was sought to fill this gap in knowledge by systematically characterizing factors that make efficacious lower airway probiotics that can competitively exclude Burkholderia from the lung environment. Specifically, it was hypothesized that negative microbial interactions between a probiotic and pathogen better protect the host against infection. The present systematic study of the microbial ecology of these organisms enables development of a pipeline for nomination of effective single and multi-organism probiotics that, to this point, remain underdeveloped. It was shown that using this pipeline, it was possible to accurately identify pairwise combinations of organisms with synergistic activity against Burkholderia. Finally, the ability of these prophylactically administered probiotics to promote survival during a challenge with a model pathogen, Burkholderia thailandensis, in vivo was tested.

### Results:

### Isolation and Characterization of Candidate Probiotics

To develop a system for testing the efficacy of lower airway probiotics in preventing infection, it was first necessary to identify a suitable pathogen to investigate. Burkholderia thailandensis (Bt) is a pathogen of mice that is genomically similar to several human pathogens including organisms of the Burkholderia cepacia complex which are common causes of death in people with cystic fibrosis (CF). Thus, Burkholderia thailandensis is an appropriate subject of study for our purposes as it is safe, infectious in mice and a good model of several pathogens which are common in those with a history of respiratory disease.

To acquire candidate probiotics for use in the lower airway, organisms were sought that would be able to survive in the lower airway for an extended period of time and be capable of tolerating the relatively low nutrient environment of the lung. It was hypothesized that organisms that were isolated directly from the lung would fulfill this criteria as they are already well adapted to the environment and may be more immunologically tolerated by the host.

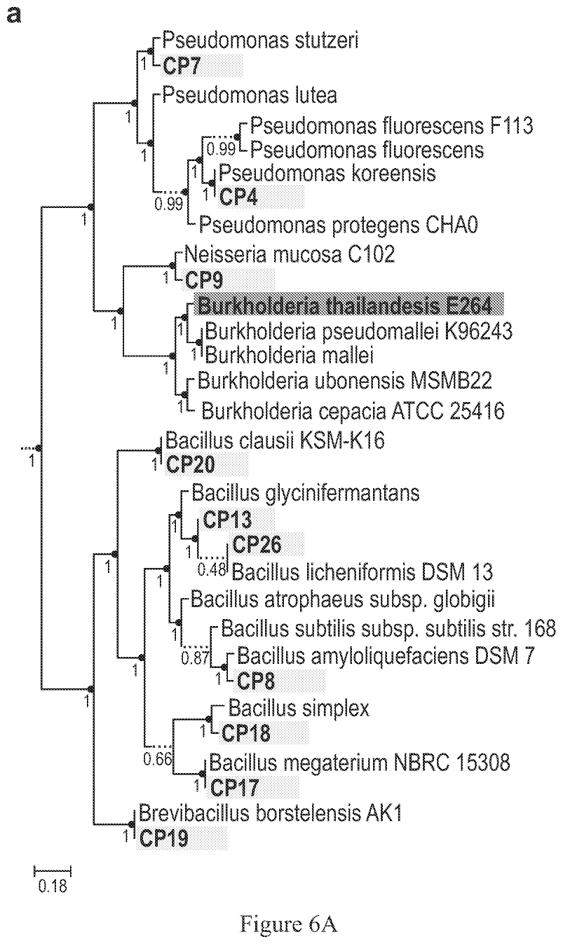

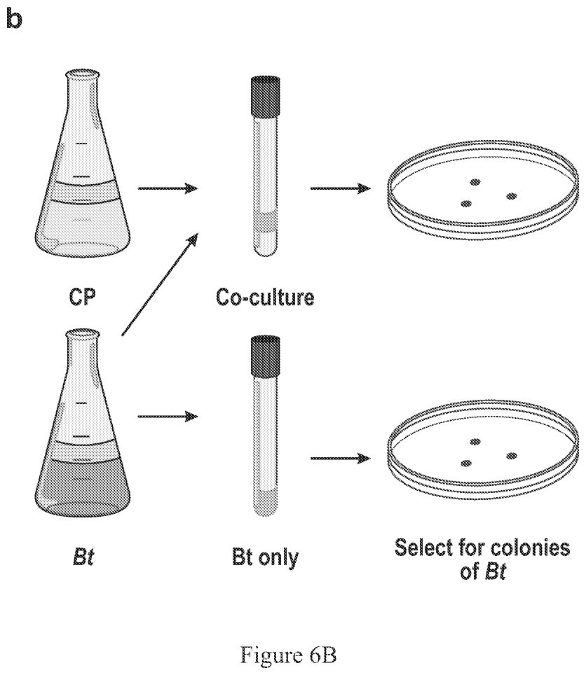

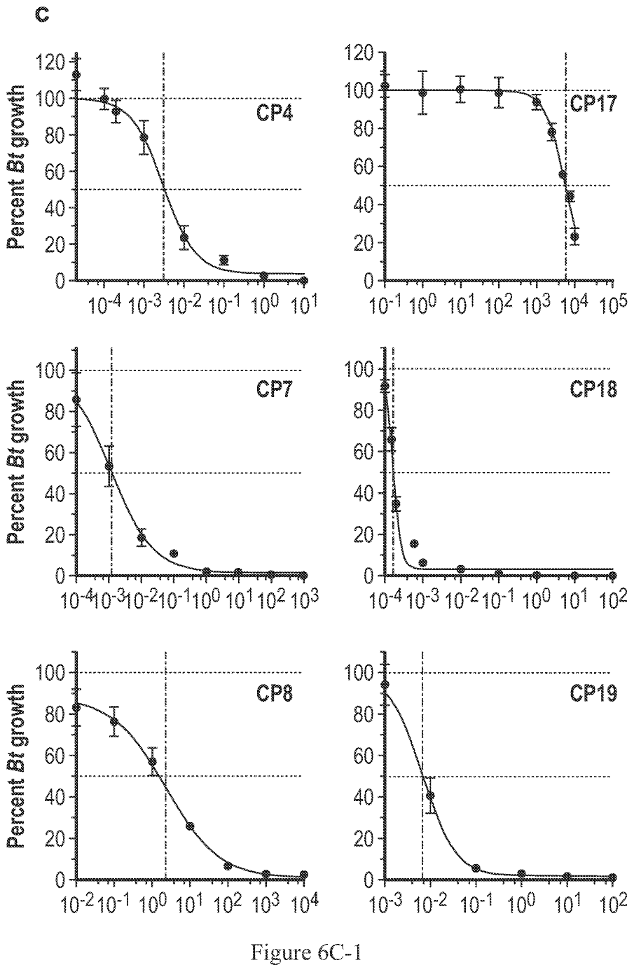

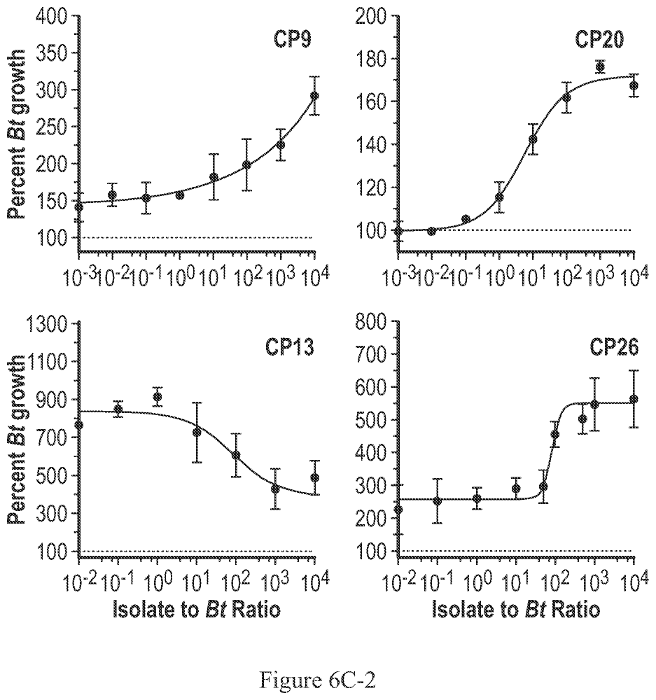

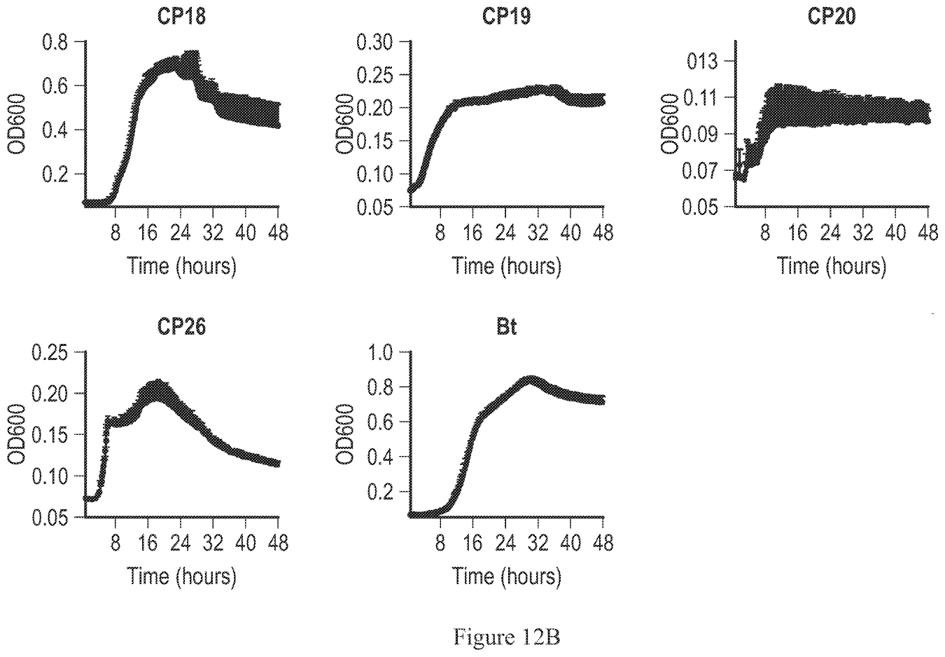

To isolate candidate probiotics, lungs from conventional C57/B16 mice were harvested, homogenized and plated onto LB agar. Plates were periodically monitored for growth and colonies were harvested for isolation and whole-genome sequenced. Prior work on the lung microbiome has led to the widely held hypothesis that many organisms that are identified are unable to actively replicate but are instead largely dormant or simply transient colonizers from the nose and mouth. It was sought, therefore, to select for organisms that would be able to actively grow in the lung with the supposition that those that were metabolically active in the lung would be able to better compete with an invading pathogen and engraft. To do this, all previously mined isolates were grown in lung simulating medium (LSM). LSM is a growth medium designed previously based on compositional analysis of sputum samples from cystic fibrosis and is thus a similar nutritional environment to what native microbiota encounter. Overall, 10 isolates were able to experience greater than one doubling in the medium after 48 hours (FIG. 12). These organisms and model pathogen Bt are arrayed on a phylogenomic tree (FIG. 6A). The organisms isolated from the lung are henceforth called candidate probiotics (CPs).

Next, the aim was to identify those organisms that were able to compete against Bt with the hypothesis that better competitors would be more efficacious probiotics. Expression of competitive phenotypes are dependent on several factors including the cell density relative to the available resources. Therefore, it was decided to assay competition across varying cell densities with the hypothesis that this would enable us to capture information about CP potency. Each CP was co-cultured with Bt at increasing concentrations of CP using competition assays (FIG. 6B). Overall, the CPs show a range of activities from negative interactions (FIG. 6C, top 6 plots) to positive interactions (FIG. 6, bottom 4 plots). Additionally, CPs demonstrate different potency as indicated by IC50 value (Table 4).

TABLE 4
Species Names and Relative IC50 Values for Candidates Probiotics

| | | | 95% Confidence |
|---|---|---|---|
| CP | Species | Relative IC50 | Interval (CI) |
| CP4 | Pseudomonas fluroescens | 0.003086 | 0.001791-0.005331 |
| CP7 | Pseudomonas stutzeri | 0.001172 | 0.0006442-0.003346 |
| CP8 | Bacillus velezensis | 2.316 | 0.8280-5.073 |
| CP9 | Neisseria lactamica | Non-inhibitory* | N/A |
| CP13 | Bacillus licheniformis | Non-inhibitory* | N/A |
| CP17 | Bacillus megaterium | 5738 | 3598-ND** |
| CP18 | Brevibacterium frigoritolerans | 0.0001693 | 0.0001633-0.0001850 |
| CP19 | Brevibacterium borstelensis | 0.00604 | 0.0064842-0.011411 |
| CP20 | Bacillus clausii | Non-inhibitory* | N/A |
| CP26 | Bacillus licheniformis | Non-inhibitory* | N/A |

*Non-inhibitory CP do not have relative IC50 values associated with them
**value could not be determined

### Investigation of Interference Competition

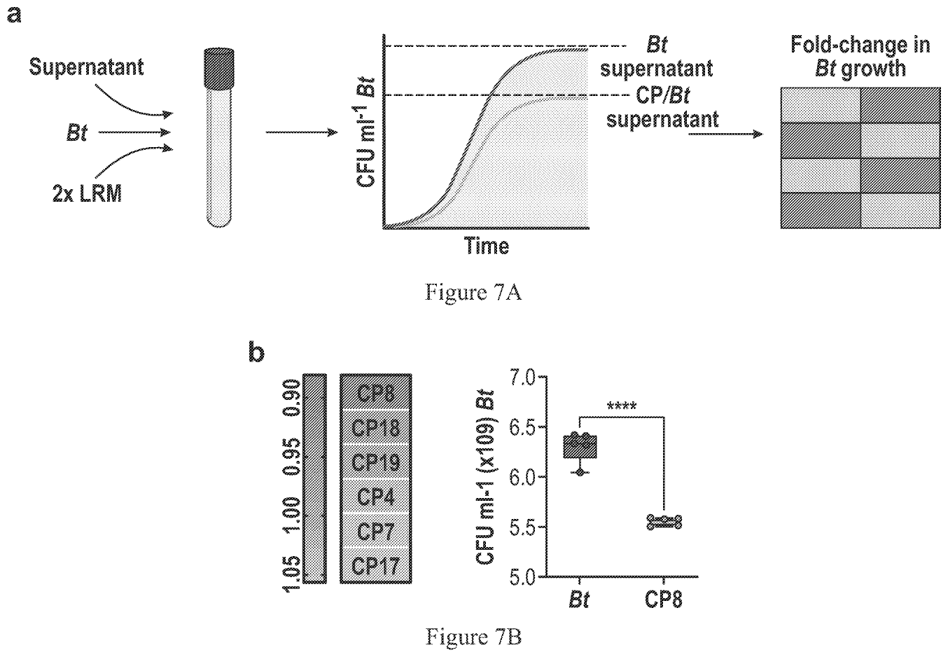

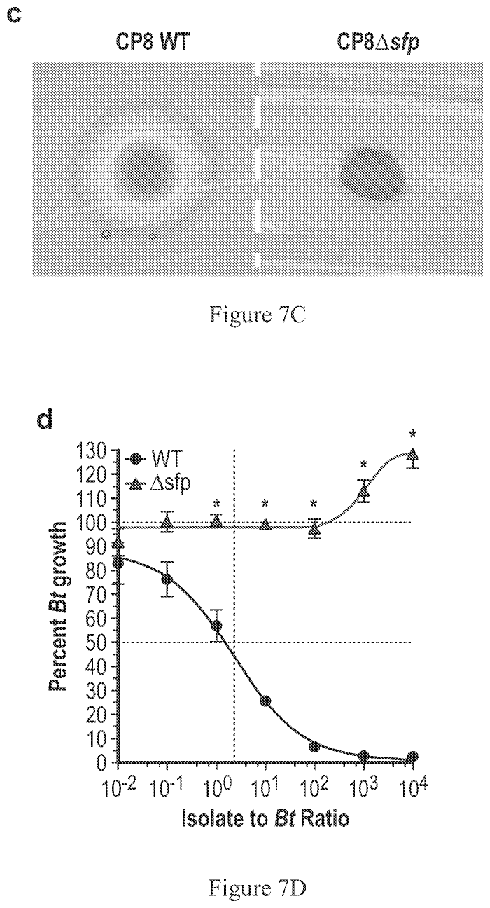

Next, it was sought to better understand the mechanism of the observed negative interactions. It was hypothesized that negative interactions could be facilitated by direct chemical attack (interference competition) or indirectly through resource consumption (exploitative competition). To assay for the production of specialized metabolites, supernatant from each of the CPs grown in co-culture with Bt was tested (FIG. 7A). It was predicted that if Bt-inhibiting secondary metabolites were produced by a given CP, then growth of Bt would be inhibited when grown in co-culture supernatant. Supernatant from one isolate, CP8, significantly reduced growth of Bt compared to a Bt-only supernatant control (FIG. 7B).

To understand the degree to which CP8's secondary metabolite is responsible for mediating the competitive phenotype observed in FIGS. 6A-6C, the aim was to create a knockout of this molecule in CP8. From prior analyses, CP8 is predicted to encode production of 22 specialized metabolites. The synthesis of all of the top hits is dependent on sfp, a phosphopantetheinyl transferase that catalyzes conversion into their active holo-form. Therefore, it was hypothesized that knocking out sfp from CP8 would result in an isolate with reduced inhibitory activity against Bt. A clean deletion of the sfp gene was made in CP8 and an agar diffusion assay was performed which showed a reduction in the zone of inhibition indicating that interference competition was reduced (FIG. 7C). Finally, a competition assay was performed using the CP8Δsfp mutant. The mutant showed reduced inhibitory activity compared to wildtype CP8 and, in contrast, demonstrated a positive interaction with Bt when interference competition was disrupted (FIG. 7D).

### Investigation of Niche Overlap as a Predictor of Competition

While interference competition appeared to be the primary mode of inhibition for CP8, five other CPs showed a negative interaction with Bt did not appear to produce specialized metabolites. It was hypothesized that inhibition with these 5 isolates was due to exploitative competition whereby the passive consumption of a limiting resource by the CP would reduce the supply to Bt.

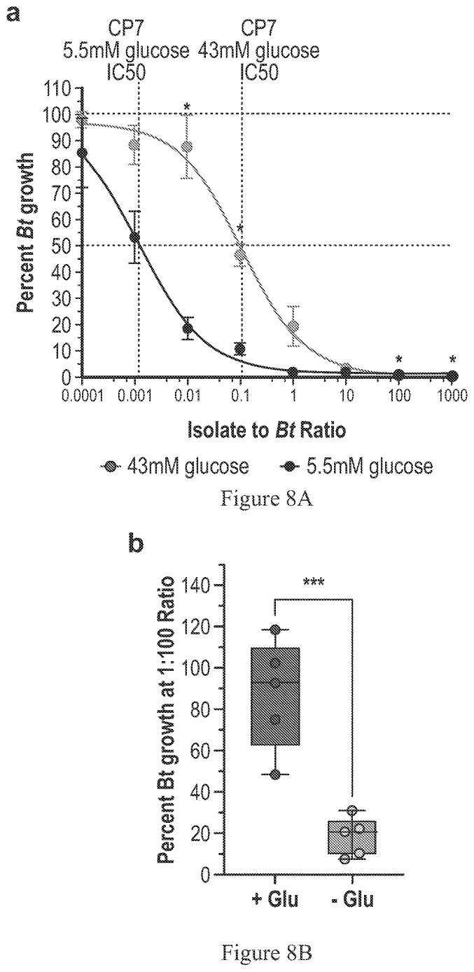

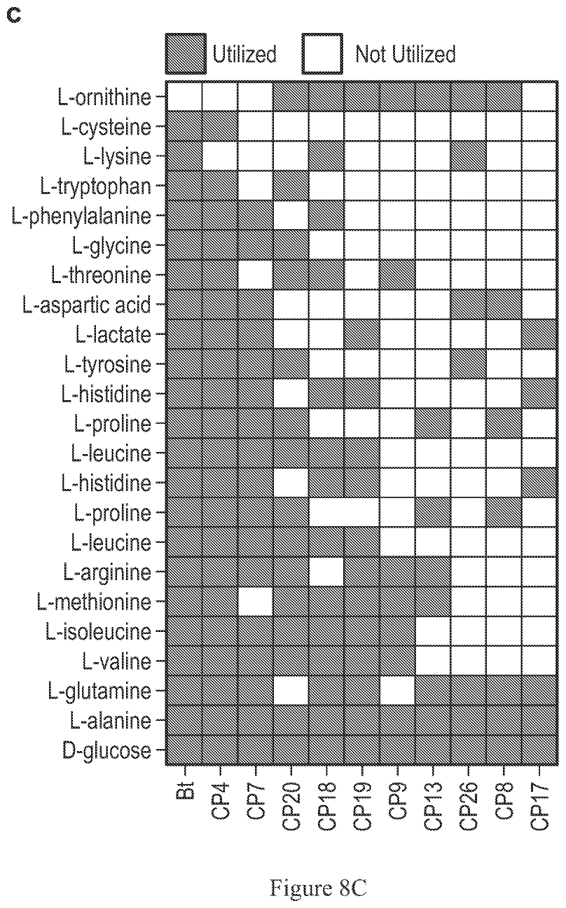

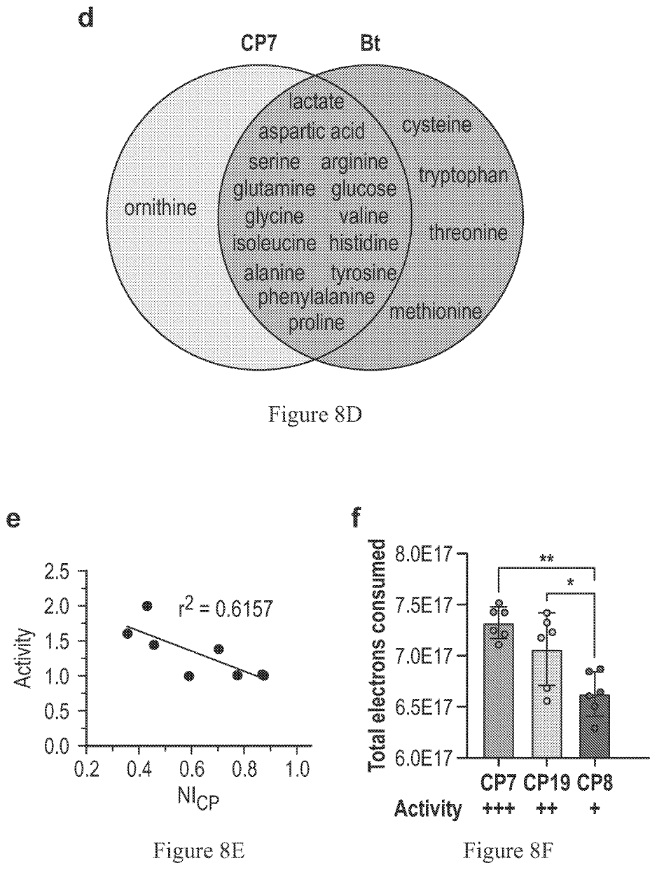

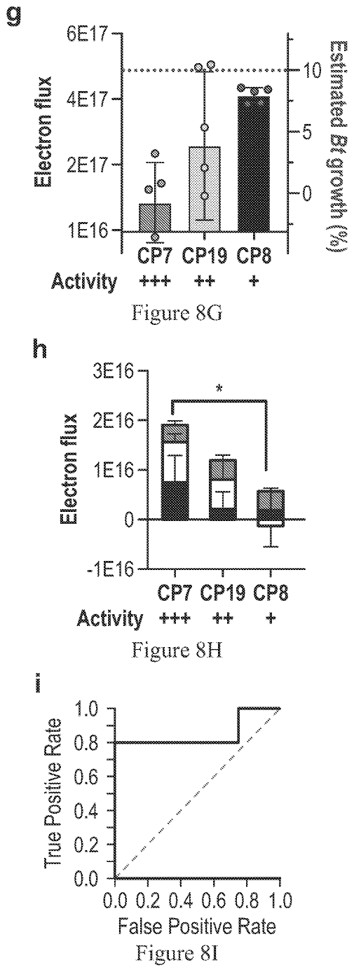

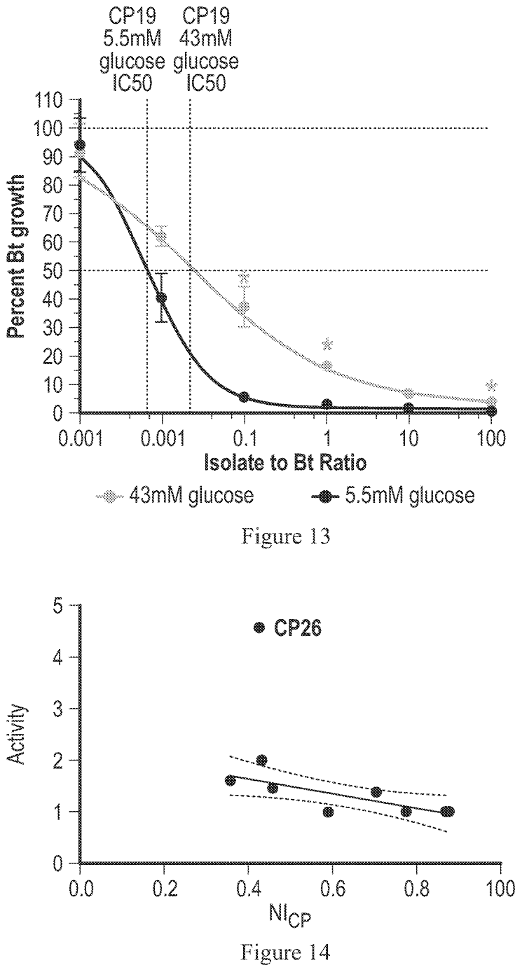

The Redfield Ratio describes the stoichiometric relationship between carbon, nitrogen and phosphorous which supports life on earth and is canonically 106:16:1. LSM has a C:N:P ratio of approximately 88:14:1, which indicates that carbon may be the limiting nutrient and thus would be competed for by the CPs and Bt. It was predicted that if carbon was limiting, adding carbon to LSM would reduce competition. To test this, LSM was supplemented with 43 mM glucose to increase the C:N:P ratio to 101:14:1. In FIG. 8A, it was observed a leftward shift of the dose-response curve for CP7 in the additional carbon condition and a significant reduction in inhibition at a 1:100 ratio (FIG. 8B). A similar result is observed when the same experiment is conducted in CP19 (FIG. 13). This result suggests that there is a reduction in competition with increased carbon abundance and indicates that carbon is the primary nutrient for which CPs and Bt are competing.

Further, it was sought to explore the extent to which competition for carbon with Bt could predict inhibitory capabilities of each CP. To do this, the growth of each CP and Bt in each individual carbon source from LSM was assayed to determine which carbon sources could be utilized by each organism (FIG. 8C). Bt shows a wide range of metabolic potential and is able to grow on every carbon source presented except for ornithine. This is perhaps unsurprising as it has been shown previously that pathogens acquire new metabolisms in order to survive in their host. Conversely, the CPs display diverse metabolic activity with CP4 and CP7 appearing most similar (FIG. 8C).

It was reasoned that those CPs that were capable of competing for more carbon-derived electrons with Bt would have more potent inhibition against the pathogen (FIG. 8D). On this assumption, a simple index of niche overlap that weights each carbon source in LSM utilized by a given CP by the theoretical electron contribution the source can make based on its oxidation-reduction half-reaction was built. It is calculated as follows:

For all carbon sources in LSM, E is a vector which holds the total electron contribution of each carbon source in the media. These values are based on concentration and redox half-reaction:

$$\vec{E} = ( \begin{matrix}glucose \\ lactate \\ asparticacid \\ threonine \\ serine \\ glutamine \\ proline \\ glycine \\ alanine \\ cysteine \\ valine \\ methionine \\ isoleucine \\ leucine \\ tyrosine \\ phenylalanine \\ ornthine \\ lysine \\ histidine \\ tryptophan \\ arginine\end{matrix} ) ,$$

The weighted value for each carbon source is:

$$\vec{W} = \frac{\vec{E}}{{E}_{glucose}} \qquad ( 1 )$$

For all CPs and Bt, a carbon utilization vector is generated. If a carbon source is consumed, then i=1, if it is not able to be consumed, then i=0.

$$\vec{{CU}_{CP}} = [ \begin{matrix}{i}_{1} \\ 2 \\ {i}_{3} \\ {i}_{4} \\ ⋮ \\ {i}_{n}\end{matrix} ] ,$$

To calculate the niche index of each CP with Bt, the equation is:

$${NI}_{CP} = \frac{[ C {\vec{U}}_{CP} ⊙ C {\vec{U}}_{Bt} ] \cdot \vec{W}}{[ C {\vec{U}}_{Bt} \cdot \vec{W} ] - [ {CU}_{CP , orn} \cdot W ]} \qquad ( 2 )$$

Niche index is higher when a given CP shares a greater number of electrons with Bt (Table 5). Furthermore, niche index appears to be a good predictor of inhibitory activity against Bt at a 1:1 ratio (FIG. 8E). Notably, CP8 is excluded from this analysis as its potency is expected to be primarily a result of secondary metabolite production rather than carbon consumption. Additionally, robust regression analysis showed CP26 as an outlier and thus it is not included in the analysis (FIG. 14).

TABLE 5
CP Activity and Niche Index Values for Candidate Probiotics

| CP | Bt growth at a 1:1 ration (%) | Niche Index |
|---|---|---|
| CP7 | 1.89 | 0.875 |
| CP4 | 3.20 | 0.869 |
| CP19 | 3.05 | 0.775 |
| CP17 | 98.99 | 0.704 |
| CP18 | 0.53 | 0.591 |
| CP20 | 115.19 | 0.457 |
| CP26 | 259.99 | 0.433 |
| CP13 | 913.04 | 0.421 |
| CP9 | 157.143 | 0.357 |
| CP8 | 56.97 | 0.351 |

As the design of the niche index relies on growth measures, it was also desired to know if better competitors were also better consumers. To confirm this hypothesis that those organisms that compete for more carbon-derived electrons with Bt are better competitors, exometabolomics was performed on supernatant from three CPs. These three CPs represent a range of inhibition strengths and niche indexes, with CP7 being the most potent (IC50=0.001172 Ni=0.875), CP19 displaying moderate potency (IC50=0.006604 Ni=0.775) and CP8 being the least potent (IC50=2.316 Ni=0.351). Each of these CPs was combined with Bt at their respective IC90 and co-incubated for 36 hours. Supernatant from these CPs at 36 hours show that more potent CPs consume a statistically greater amount of carbon from the media than less potent CPs (FIG. 8F). At 24 hours where the net effect of each of the CPs on Bt is the same, better competitors achieve Bt suppression by consuming fewer carbon-derived electrons than worse competitors (FIG. 8G). This led to the hypothesis that certain carbon sources were being prioritized allowing better competitors to have the same net effect on Bt while consuming fewer electrons. Further, it was speculated that if high priority carbon sources were to be identified, perhaps the consumption of a smaller set of these high priority carbon sources could predict activity with similar accuracy to the niche index.

From the exometabolomics data, consumption of three carbon sources, lactate, proline and aspartic acid are prioritized more at 24 hours by the most potent candidate probiotic, CP7, than the least potent, CP8 (FIG. 8H). Furthermore, it was found that a CPs ability to generate biomass on lactate was a good predictor of its ability to inhibit the pathogen suggesting that the ability to consume lactate in co-culture may be important for pathogen control (FIG. 8I). Overall, these results demonstrate that different resources may have different competitive effects on an interaction.

### Niche Overlap Predicts Combination Efficacy

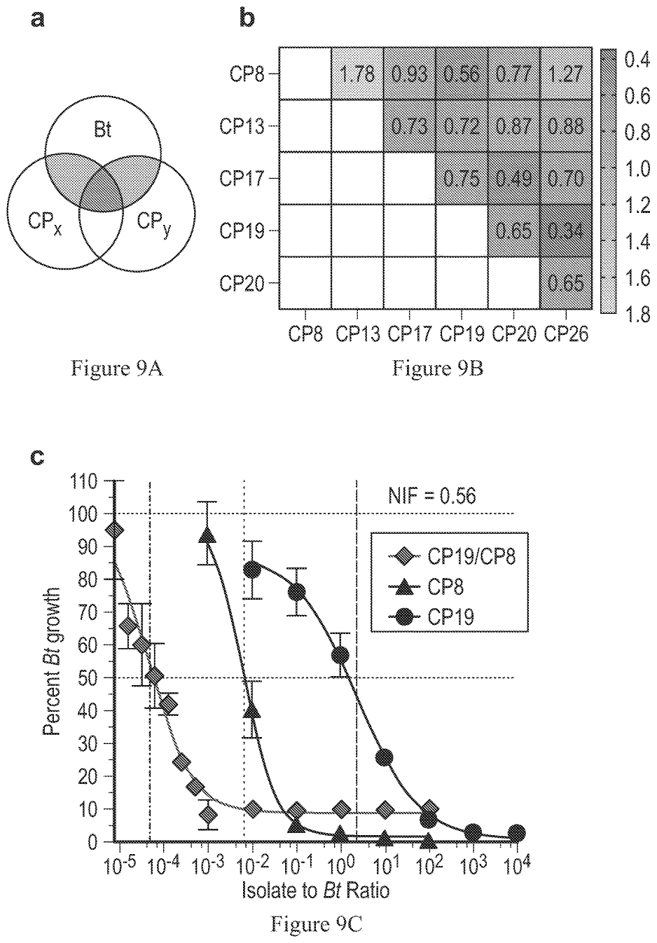

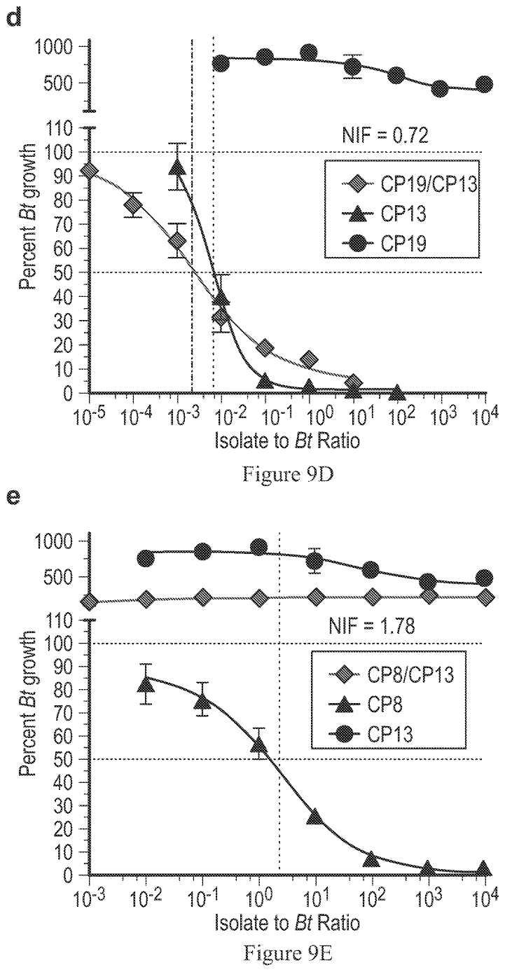

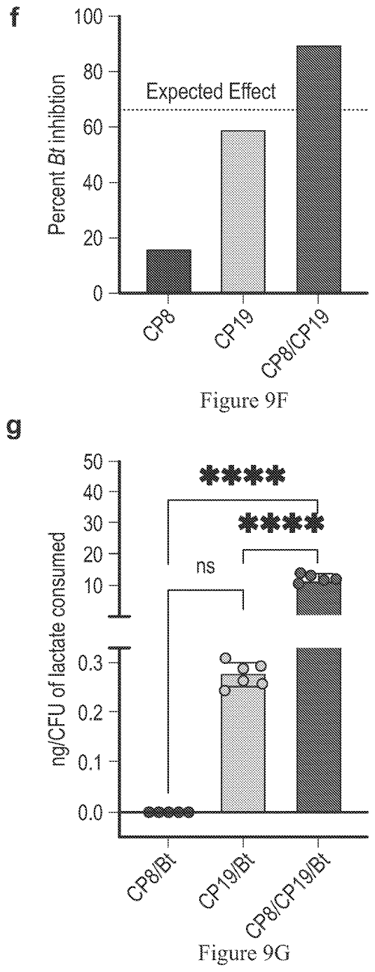

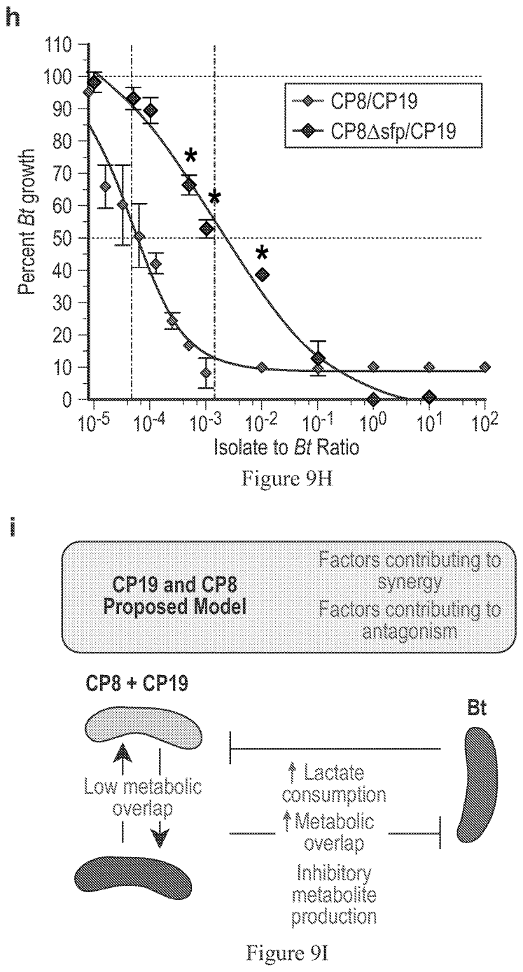

While the work in FIGS. 6A-6C showed that a few of the CPs were potent inhibitors of Bt in individual co-culture, many were poor inhibitors or promoted growth of Bt. Having found that niche index was a good predictor of individual CP potency in vitro, it was hypothesized that this metric could be altered to identify pairwise combinations of poor-performing CPs with enhanced antagonism. To do this, a new metric was created, the niche index fraction, which is composed of the niche overlap between two given CPs divided by the niche overlap between these two CPs and Bt (FIG. 9A). It is calculated as follows:

First, the overlap between two CPs is generated and is weighted by the relative electron contribution of each carbon source

$$y = [ {\vec{CU}}_{CPx} ⊙ {\vec{CU}}_{CPy} ] \cdot \vec{W} \qquad ( 3 )$$

Total carbon-derived electron utilization is calculated for both CPs

$$p = {\vec{X}}_{CPx , CPy} \cdot \vec{W} , such that p is never 0 \qquad ( 4 )$$

For which

$${X}_{Cpx , CPy} = \{ \begin{matrix}{CU}_{i , CPx} = 1 = {CU}_{i , CPy} ; 1 \\ {CU}_{i , CPx} = 0 = {CU}_{i , CPy} ; 0 \\ {CU}_{i , CPx} \neq {CU}_{i , CPy} ; 1\end{matrix} \qquad ( 5 )$$

Thus, the niche overlap between two CPs is

$${NI}_{Cpx , CPy} = \frac{y}{p} \qquad ( 6 )$$

The niche overlap between two CPs and Bt is thus

$${NIF}_{Cpx , CPy} = \frac{{NI}_{Cpx , CPy}}{[ C {\vec{U}}_{Bt} \cdot \vec{W} ] - [ {\vec{X}}_{CPx , CPy , orn} \cdot \vec{W} ]} \qquad ( 7 )$$

NIF was calculated for every pairwise combination of the seven worst Bt performers from FIGS. 6A-6C. This generated a matrix of potential synergistic combinations (FIG. 9B). It was hypothesized that combinations with lower niche index fractions would indicate high niche overlap with Bt but low niche overlap between two isolates and were thus more likely to act synergistically to inhibit Bt. To test the ability of this metric to predict CP interactions, pairwise combinations with the three lowest fractions as well as a combination with a moderate fraction and high fraction using competition assays were chosen to test.

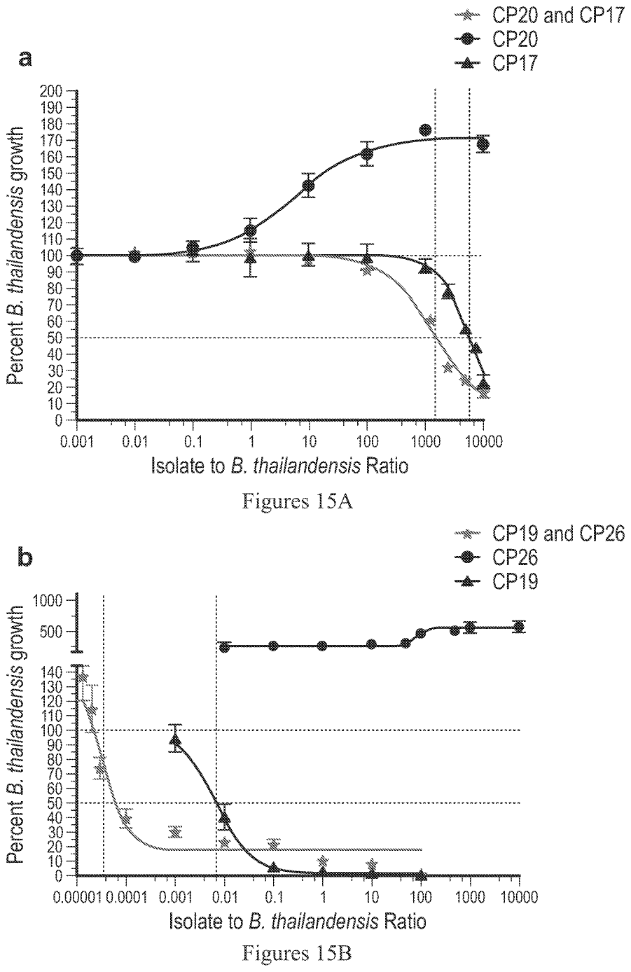

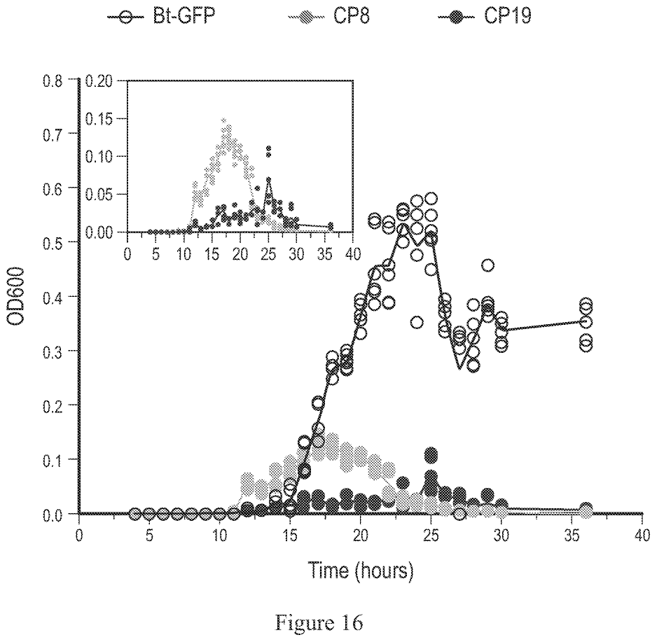

In these assays, two CPs were combined at a 1:1 ratio such that the total CP concentration at a given ratio was the same as their individual CP counterparts. This enabled a direct comparison of activity of an individual CP and the activity of a combination of two CPs at a given ratio as the total density was the same in each. From competition assays, it was determined that niche index fraction was able to predict synergistic combinations and non-synergistic combinations as reflected by their relative IC50 values (FIGS. 9C-9E and Table 6). Further, the combination with the largest leftward shift, CP19 and CP8 (FIG. 9C) was determined to be synergistic via the bliss independence model (FIG. 9F). Several other combinations with low niche index fractions showed similar results, however, bliss independence index was only calculated in cases where both CPs individually demonstrate an inhibitory effect (FIGS. 15A-15B).

TABLE 6
Relative IC50 hillslope and values and
niche fraction for CP combinations tested

| | | | Niche | |
|---|---|---|---|---|
| | | Hill | Index | |
| CP | Relative IC50 | coef- | Fraction | |
| Combination | (95% CI) | ficient | (N_{IF}) | FIG. |
| CP19/CP26 | 3.528 × 10⁻⁵ | 1.213 | 0.34 | FIG. 15A |
| | (2.576 × 10⁻⁵-5.105 × 10⁻⁵) | | | |
| CP17/CP20 | 1501 | 0.063 | 0.49 | FIG. 15b |
| | (1071-1935) | | | |
| CP8/CP19 | 4.707 × 10⁻⁵ | 0.761] | 0.56 | FIG. 9C |
| | 3.256 × 10⁻⁵-9.914 × 10⁻⁵) | | | |
| CP19/CP13 | 0.002126 | 0.405 | 0.72 | FIG. 9D |
| | (0.0007314-0.008040) | | | |
| CP8/CP13 | Non-inhibitory* | N/A | 1.78 | FIG. 9E |

*Non-inhibitory combinations do not have relative IC50 values associated with them

For one combination, CP8 and CP19, it was sought to understand the mechanism by which competition was being enhanced. First, to understand the effect of the interaction on all members of the co-culture, growth curves of each individual strain in the CP8, CP19 and Bt when in co-culture were performed. Reduced growth in co-culture of all three organisms classifies this antagonistic interaction as competition. To better understand how CP8 and CP19 may enhance each other's antagonistic impact on Bt, once again, exometabolomics analysis on supernatant from this combination was performed. At 24 hours, significantly more lactate was consumed per cell in the combination than by CP8/Bt co-culture or CP19/Bt co-culture (p<0.0001) (FIG. 9G). Similarly, to the single isolate studies, this result once again suggests that lactate utilization may be important for competition with Bt in a lung-like environment.

To further investigate how CP8 and CP19 act synergistically, a competition assay was performed using the CP8Δsfp mutant in combination with CP19 to understand the degree to which secondary metabolite production was responsible for this synergistic effect (FIG. 9H). When wildtype CP8 was replaced with the Δsfp mutant, there was a significant reduction in Bt inhibitory activity compared to the wildtype CP8/CP19 combination. Together, these findings suggest that both carbon consumption and secondary metabolite production are important for activity of the CP8/CP19 combination and likely enable Bt inhibition activity (FIG. 9I).

### CPs Provide Protection Against Infection

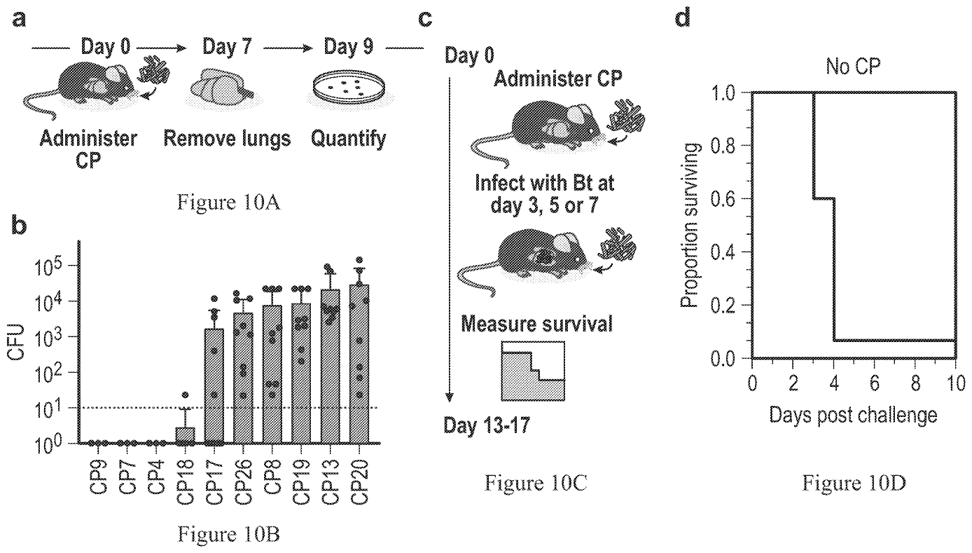

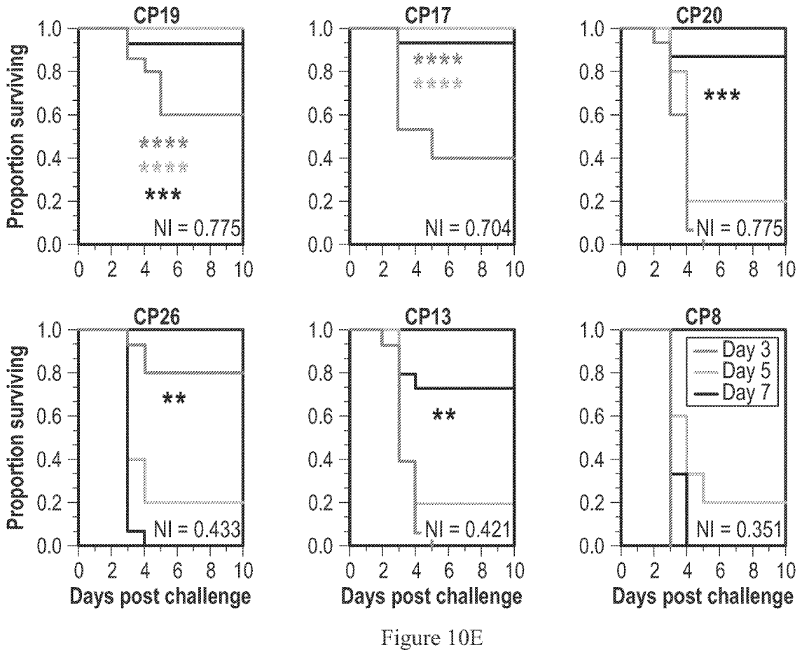

Having observed the ability of the CPs to compete with Bt in an in vitro lung-stimulating environment, it was decided to test their ability to confer protection against the pathogen in vivo. First, it was hypothesized that those CPs which engrafted at high density over an extended period of time would serve to better protect against infection. To test the engraftment capabilities of each CP, 10⁶ CFU of each CP was administered via oropharyngeal administration (OPA) to C57BL/6 mice and waited seven days to allow engraftment to occur before removing the lungs to quantify the bacterial load (FIG. 10A). Overall, several CPs showed the ability to engraft in the lung for 7 days (FIG. 10B). CPs whose mean CFU load in the lung was above 10 CFU were particularly interesting and it was hypothesized that these organisms would be most likely to protect against pathogen invasion.

While it was hypothesized from the in vitro work that direct competition between the CP and pathogen would confer the strongest protection against pathogen infiltration, immune modulation is also a potential mechanism by which probiotics have been hypothesized to prevent infection. Therefore, ten CPs were screened for their ability to promote survival regardless of engraftment capabilities. Each of these ten CPs or saline were administered to the lungs of mice as previously described. Mice were subsequently challenged with Bt 3, 5 or 7 days after CP administration and survival was measured for 10 days following challenge (FIG. 10C). Mice administered saline show poor survival with few mice surviving past day 4 (FIG. 10D). In contrast, mice show improved survival when prophylactically dosed with certain CPs (FIG. 10E). While several CPs are protective when administered 3 days prior to Bt challenge, two CPs CP19 and CP17 show significantly enhanced survival at several dosing intervals. CP17 is protective when administered 3 and 5 days prior to pathogen challenge while CP19 is efficacious at every interval tested.

It was observed that these most protective probiotics, CP19 and CP17, were predicted to have the highest niche overlap with Bt via their niche index values and show an ability to inhibit in competition assays (FIG. 6C). Furthermore, the candidate probiotics that do not inhibit Bt in competition assays but do readily engraft (FIG. 10B) (CP13, CP20 and CP26) show no significant inhibition of Bt at day 5 and 7 post-inoculation. A notable exception to this is CP8, which shows inhibition in vitro and can engraft but is not able to protect mice from death due to subsequent infection. This suggests that resource competition may be more predictive of the protective capabilities of a probiotic than secondary metabolite production.

### Discussion:

The human lung microbiome composition has been linked to both respiratory health and the occurrence of respiratory tract infections. As a result of this connection, manipulation of the lower airway microbiome is an attractive target for therapies. Manipulation of the lung microbiome via supplementation with probiotics has shown success in preventing lung metastases and pneumococcal infection. However, these studies lack a more broad and systematic analysis which is necessary to learn generalizable principles for the design of efficacious lower airway probiotics and guide future development for a variety of conditions.

In this work, a model system was developed that probes interactions between host-derived candidate probiotics (CPs) and a model pathogen Burkholderia thailandensis. From this exploration, it was found that resource competition is the most frequent antagonistic phenotype in interactions between CPs and Bt in vitro. Specifically, competition for carbon seems to play an important role in these interactions. As a result, niche overlap is indicative of CP inhibitory activity in vitro. It was proposed that the consumption of certain carbon sources may be a good indicator of the inhibitory capabilities of a given CP. Furthermore, these same principles were used to design combinations of CPs with enhanced antagonism against Bt. The mechanism by which one combination, CP19 and CP8, exploits both resource competition and inhibitory metabolite production to its advantage in competition with Bt was then explored. Finally, it was uncovered that several CPs are able to engraft in lungs in vivo for out to 7 days and that two CPs, CP19 and CP17, significantly improve survival outcomes after Bt challenge. Thus, niche overlap appears to have some connection to protection in vivo.

Currently, probiotics are tested for clinical applicability using extensive in vitro and in vivo screening methods which are costly and time-consuming. In the present work, it was found that one way that multi-organism probiotics defend against a pathogen is by the consumption of various glycolytic substrates. In particular, lactate consumption appears to be important for efficacy. Several studies have found that there is increased lactate in the lung during inflammatory exacerbations which may feed pathogen growth.

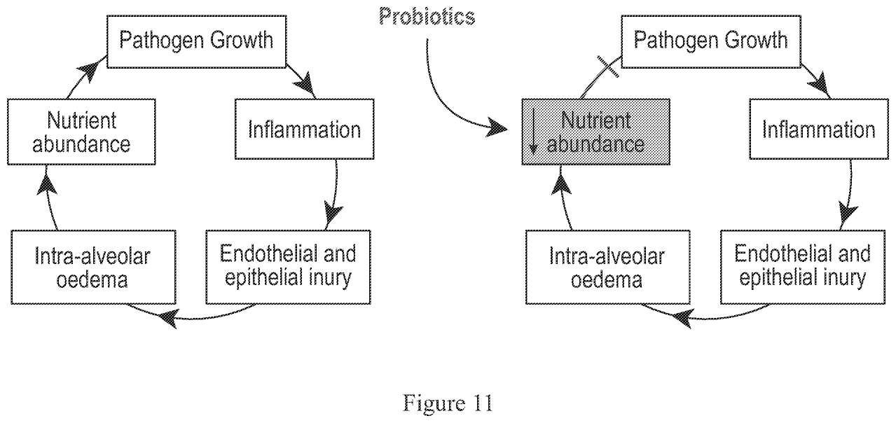

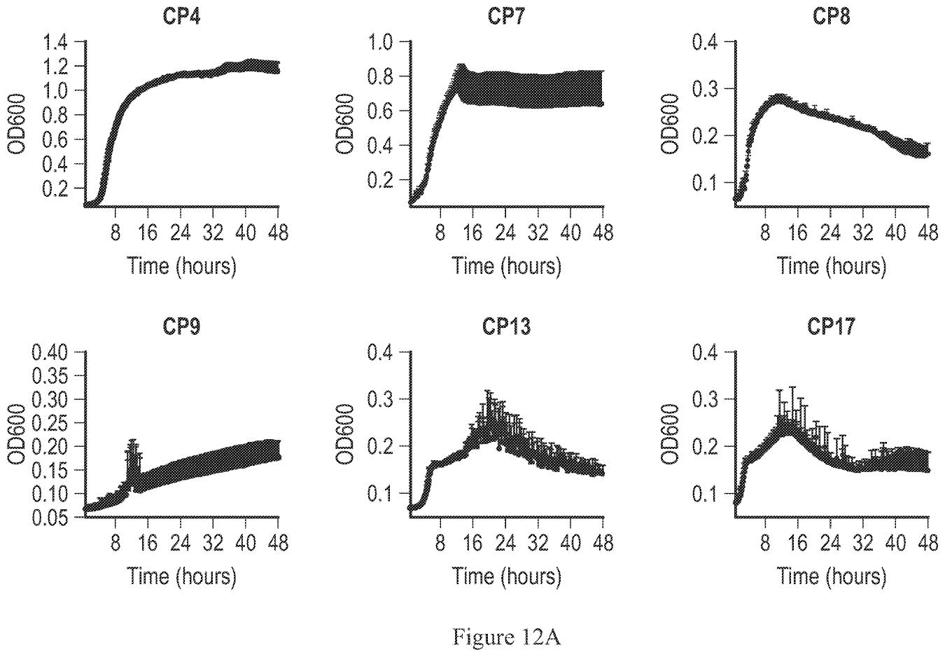

Broadly, it was proposed that lower airway probiotics may have functionality by stopping the positive feedback loop of infection. In this model, pathogen infiltration into the lung and growth results in inflammation and cellular injury causing a leak of nutrient rich fluids into the alveolar compartment further promoting pathogen growth (FIG. 11 (left)). Lower airway probiotics might stop this feedback loop by depleting nutrient abundance in the local lung environment thus limiting pathogen growth (FIG. 11 (right)).

### STAR Methods:

### Isolation and Culture of Bacteria

A Burkholderia thailandensis E264 strain that constitutively expresses GFP was provided by Daniel J. Hassett (University of Cincinnati College of Medicine; Cincinnati, OH). The Candidate Probiotic (CP) isolates were recovered from the lower respiratory tracts of healthy mice using a culturomics approach. All animal work was conducted in accordance with protocols approved by the Lawrence Livermore National Laboratory (LLNL) Institutional Animal Care and Use Committee (IACUC, protocol 304) and Institutional Biosafety Committee (IBC, protocol 2021-010). LLNL is accredited by the Association for Assessment and Accreditation of Laboratory Animal Care (AAALAC) International, and is Public Health Service-Assured (PHS assurance A3184-01). Briefly, airway (trachea and lung) tissues were collected from 24 mice, homogenized in 500 μL of phosphate-buffered saline (PBS), and used to inoculate five different solid growth media [tryptic soy (TS); TS+5% sheep's blood; M9 minimal salts+0.2% glucose; brain-heart infusion (BHI); and lysogeny broth (LB) (Teknova; Hollister, CA)] as well as three different BACTEC blood bottles [Plus Aerobic/F; Plus Anaerobic/F; and Lytic/10 Anaerobic/F (Becton Dickinson; Franklin Lakes, NJ)]. The inoculated solid growth media were incubated at 37° C. for seven days. The inoculated blood bottles were incubated with shaking (250 rpm) for 1-5 days, and 100 μL aliquots were periodically withdrawn for use in inoculating the five different solid growth media, which were then incubated at 37° C. for seven days. All colonies detected on the solid growth media were individually transferred to LB agar and, after further incubation at 37° C. for 1-3 days, streaked to single colonies on fresh LB agar. A representative single colony from each Petri plate was used to inoculate LB liquid and solid media, and these cultures incubated at 37° C. for 1-3 days for use in preparing frozen glycerol stocks as well as extraction of genomic DNA for sequencing.

### Genome Sequencing and Analysis

Genomic DNA extracted from each CP isolate was sequenced using an Illumina NextSeq 550 instrument (150SE reads). Sequence assemblies were generated using BBDuk and SPAdes, and aligned against genome sequences in RefSeq for phylogenetic placement. All phylogenetic placements were confirmed using autoMLST as well as a program called Speciate, which applies criteria (ANI similarity and circumferences to species representatives) of the GTDB system.

### Lung Simulating Medium (LSM) Preparation

LSM was made according to Palmer (2006). Adjustments were made to the original formulation to increase sodium and chloride concentrations to more closely match those in healthy lungs. As a result, LSM contains 92 mM NaCl instead of 66.6 mM in SCFM⁶.

### Competition Assays

3 ml overnight cultures were prepared at 37° C. and 278 rpm of individual CPs and Bt in LSM from a stab of glycerol stock. After incubation for 24 hours, cultures were diluted again in LSM at 1:50 and grown until late log phase. All cultures were spun down and resuspended in fresh LSM. CPs were diluted according to the ratios being tested where a 1:1 ratio represented a cell suspension of 3.33E4 CFU/mL of CP, 0.1 ratio represented 3.33E3 CFU/mL etc. After dilution, 3.33E4 CFU/ml of Bt was added to each 15 ml CP dilution tube and a Bt-only control. The 15 ml volume was split into 5-3 ml to make biological replicates. All samples were incubated at 37° C. overnight with shaking at 278 rpm.

After 24 hours, samples were removed and diluted 1:100-1:100,000 in PBS and plated onto LB carbenicillin100 to select for colonies of Bt. Plates were incubated for ~48 hours at 37° C. or until colonies were visible. Quantification was done by counting colonies. Percent Bt growth was calculated by taking the CFU/ml of each replicate culture and dividing by the average of the CFU/ml in the Bt-only control and multiplying by 100. Dose-response analysis was performed using the ECAnything algorithm in Graphpad Prism 10.

### Supernatant Inhibition Experiments

CPs and Bt were grown overnight in LSM as previously described. Cultures were diluted to 5E5 CFU/ml and each CP was combined with Bt, a Bt-only control was also grown. After ~72 hours of growth, cultures were centrifuged, and supernatant was collected and sterile filtered with a 0.22 μM filter. All supernatant samples were pH-adjusted to 7.03. Twice concentrated LSM and supernatant were mixed 1:1 and exponential-phase Bt was added to each sample at 3.33E4 CFU/mL. Growth curves were performed using a plate reader measuring OD600 for 30 hours with shaking at 240 rpm and 37° C.

### CP8 Sfp Knockout

The CP8Δsfp mutant was made by the double-crossover homologous recombination method. Briefly, homologous arms were amplified up and downstream of the sfp region and cloned into the T2 (2)-ori plasmid in addition to the oriT/traJ region using golden gate assembly. The resulting plasmid was conjugated into CP8 using an established method for conjugation in Bacillus. The conjugation was plated onto LB supplemented with Kanamycin20 and Polymyxin5 to select for Bacillus colonies containing the plasmid. Colonies were outgrown in LB Kanamycin 20 broth and subsequently incubated at 45° C. on LB Kan20 agar overnight to facilitate the first crossover event. A single colony positive for the first crossover event was selected and subsequently passaged in LB broth 6 times. Replica plating was performed to check for loss of kanamycin resistance and PCR was used to screen for colonies where sfp was absent. Resulting fragments and 16S region were sequenced to confirm deletion and strain identity.

### Overlay Assays

Overnight cultures of Bt and CP8 were made in LSM as previously described. Bt was diluted to OD 0.5 and streaked onto LSM agar using a sterile cotton swab. Plates were briefly allowed to dry and 2 μl spots of CP8 overnight culture were added. Plates were incubated overnight and checked for the appearance of a zone of inhibition after 48 hours.

### Carbon Source Screening

Overnight cultures of CPs and Bt in LSM were made as previously described. Cultures were centrifuged and washed twice with 2× LSM with carbon sources removed. Cultures were diluted to an OD of 0.04 added to a 384-well plate containing all carbon sources from LSM medium at a concentration of 10 mM. Initial OD600 readings were taken using a plate reader prior to incubation. Plates were subsequently incubated at 30° C. for 3 days at 800 rpm and a plate reader was used to take their final OD600 reading. Each CP or Bt was considered able to utilize a given carbon source if the final OD600 reading was greater than the blank plus 2 standard deviations.

### Exometabolomics

Overnight cultures of CP7, CP8, CP19 and Bt were made as previously described. Cultures were backdiluted 1:50 and grown to late log phase. Cultures were made in LSM combining Bt at 3.33E4 CFU/ml with the relevant CP at the IC90 for the individual CP. Cultures were incubated at 37° C. with shaking at 278 rpm and samples were collected at 24 and 36 hours. For collection, samples were centrifuged at 3000rcf for 10 minutes. Supernatant was removed and syringe filtered to remove residual cellular material. Samples were analyzed via GC-TOF by the West Coast Metabolomics Center. Data was SERRF normalized to remove batch effect then normalized to the amount injected. Metabolite concentrations were normalized to a media-only control. For analysis, concentrations of each metabolite were converted to metabolite-contributed electrons using the oxidation-reduction half reaction for each metabolite analyzed. For carbon subset analysis, total electrons from lactate, proline, and aspartic acid in the co-culture supernatant at 24 hours were added. Electron concentrations below 0 indicate that a given metabolite is more abundant in co-culture than the media control.

### Pairwise Combination Competition Assays

Pairwise combination competition assays were performed as described in “Combination Competition Assays” with the following adjustments. After resuspension in fresh media, the two CPs of interest were mixed at a 1:1 ratio at the desired CFU/ml (eg. for a 1:1 ratio with Bt, a 3.33E4 CFU/ml CP mixture was made). Subsequent dilutions were made and carried out as previously described.

### Bliss Independence Model

The bliss independence model was used to calculate the expected combined effect of two CPs on Bt growth. The expected effect is calculated as follows:

$$Expected Effect = {E}_{A} + {E}_{B} ( 100 - {E}_{A} )$$

Where E_{A} and E_{B} are the percent inhibition of Bt when co-cultured with each CP alone. When the observed effect is greater than the expected effect, a combination is determined to have synergy. For combinations in which one or both CPs do not show inhibition in pairwise co-culture with Bt, bliss independence cannot be calculated.

### Engraftment Studies

Overnight cultures of each CP were prepared in LB broth. The next day, C57/B16 mice were inoculated with 10⁶ CFU of culture using oropharyngeal administration. Mice were monitored for 7 days for changes in body weight. After 7 days, lungs were harvested, homogenized and plated on LB agar to quantify CFU load in the lung. The livers were also removed, homogenized and plated for quantification of liver dissemination.

### Survival Studies

Mice were inoculated as described in “Engraftment Studies”. After 3, 5 or 7 days, mice were challenged with 10⁶ CFU of Bt via oropharyngeal administration. Mouse body weights and survival were monitored over the course of 10 days.

### Statistical Analyses

Ordinary one-way ANOVA with Tukey's multiple comparisons test, ROC curve analyses, Mantel-Cox survival analyses and all plotting was completed in Graphpad Prism 10.

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A pharmaceutical composition comprising a pharmaceutically acceptable carrier and a lower airway probiotic composition comprising a physiological solution and a single candidate probiotic (CP) population, wherein the CP is CP4, CP7, CP8, CP9, CP13, CP17, CP18, CP19, CP20, or CP26.

2. The pharmaceutical composition of claim 1, wherein the candidate probiotic is CP17.

3. The pharmaceutical composition of claim 1, wherein the candidate probiotic is CP19.

4. A pharmaceutical composition comprising a pharmaceutically acceptable carrier and a lower airway probiotic composition, wherein the lower airway probiotic composition comprises at least two candidate probiotic (CP) populations CP8 and CP19, CP20 and CP17, or CP19 and CP26.

5. The pharmaceutical composition of claim 4, wherein the CP population consists of CP8 and CP19.

6. The pharmaceutical composition of claim 4, wherein the CP population consists of CP20 and CP17.

7. The pharmaceutical composition of claim 4, wherein the CP population consists of CP26 and CP19.

8. The pharmaceutical composition of claim 1, wherein the pharmaceutical composition is formulated for administration to the lower airway of a subject.

9. The pharmaceutical composition of claim 8, wherein the pharmaceutical composition is formulated for administration to the respiratory tract of a subject by a dry powder inhaler or a nebulizer.

10. The pharmaceutical composition of claim 9, wherein the composition is administered using a nebulizer and the pharmaceutically acceptable carrier is a saline solution, hypertonic saline solution or water.

11. An inhaler comprising the pharmaceutical composition of claim 1.

12. A method of preventing or treating a lower respiratory infection in a mammal in need thereof comprising administering an effective amount of the pharmaceutical composition of claim 1 to the lungs of the mammal.

13. The method of claim 12, wherein the pharmaceutical composition is administered by means of a nebulizer or a dry inhaler.

14. The method of claim 12, wherein the effective amount of the CP comprises between about 0.9×10⁶ CFU to about 36.9×10⁶ CFU per day.

15. The method of claim 12, wherein the CP is administered at a total dose of about 10⁶ CFU.

16. The method of claim 12, wherein the infection is Pseudomonas aeruginosa, Mycobacteria spp, Aspergillus spp, Staphylococcus aureus, Achromobacter spp, Stenophomonas maltophilia, Burkholderia spp, Haemophilus spp, Streptococcus pneumoniae or any combination thereof.

17. The method of claim 12, wherein the infection is Burkholderia pseudomallei.

18. The method of claim 12, wherein the mammal is a human.

19. A kit comprising: at least one container and the pharmaceutical composition of claim 1, wherein the CP population reduces lower airway infection in a recipient mammal, and instructions for delivery of an effective amount of the composition to the mammal in need thereof.

20. The kit of claim 19, further comprising a nebulizer or a dry powder inhaler.
